(19) United States
(12) Patent Application Publication
(10) Pub. No.: US 2005/0158325 A1
(43) Pub. Date: Jul. 21, 2005

(54) Immunomodulatory combinations

(75) Inventors: David M. Hammerbeck, Houlton, WI (US); Ross M. Kedl, Denver, CO (US); Richard L. Miller, Maplewood, MN (US); Mark A. Tomai, Woodbury, MN (US); John P. Vasilakos, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/026,457

(22) Filed: Dec. 30, 2004

Related U.S. Application Data
(60) Provisional application No. 60/533,179, filed on Dec. 30, 2003.

(51) Int. Cl. A61K 39/395; A61K 39/00; A61K 31/4745

(52) U.S. Cl. 424/155.1; 514/291; 514/292; 514/301; 514/302; 424/184.1

(57) ABSTRACT

The present invention provides immunomodulatory combinations that includes an IRM component and a therapeutic agent, each in an amount that, when in combination with the other, is effective for inducing an immune response in a subject.

### CROSS-REFERENCE TO RELATED APPLICATION

This application claims priority to U.S. Provisional Patent Application Ser. No. 60/533,179, filed Dec. 30, 2003.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as Toll-like receptors (TLRs) to induce selected cytokine biosynthesis. They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis, multiple sclerosis), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929) and more are still being discovered. Other IRMs have higher molecular weights, such as oligonucleotides, including CpGs (see, e.g., U.S. Pat. No. 6,194,388).

In view of the great therapeutic potential for IRMs, and despite the important work that has already been done, there is a substantial ongoing need to expand their uses and therapeutic benefits.

### SUMMARY

It has been found that certain small molecule IRMs can be used in immunomodulatory combinations for treating various types of disorders. Accordingly, the invention provides immunomodulatory combinations that includes an IRM component and a therapeutic agent, each in an amount that, when in combination with the other, is effective for inducing an immune response in a subject.

In some embodiments, the IRM component can include an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In alternative embodiments, the IRM component can include a sulfonamide-substituted imidazoquinoline amine, an ether-substituted imidazoquinoline amine, a sulfonamide-substituted tetrahydroimidazoquinoline amine, an ether-substituted tetrahydroimidazoquinoline amine, a sulfonamide-substituted imidazopyridine amine, or an ether-substituted imidazopyridine amine.

In certain embodiments, the therapeutic agent can include a cancer antigen or a cancer antibody, an antigen of an infectious agent, or a medicament for treating a T_{H}2-mediated disorder.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 is a bar graph showing the lung viral titers and nasal viral titers 24 hours after viral challenge in rats, after one pre-treatment dose with an immunomodualtory combination.

FIG. 2 is a bar graph showing the lung viral titers and nasal viral titers 24 hours after viral challenge in rats, after two pre-treatment doses with an immunomodualtory combination.

FIG. 3 is a bar graph comparing the size of tumors in mice 14 days after treatment with different immunomodualtory combinaitons.

FIG. 4 is a line graph comparing tumor growth in mice after treatment with different immunomodulatory combinations.

### DETAILED DESCRIPTION OF ILLUSTRATIVE EMBODIMENTS OF THE INVENTION

Immune response modifiers (“IRMs”) include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons (e.g., IFN-α and IFN-β), tumor necrosis factor-alpha (TNF-α), certain interleukins (e.g., IL-1, IL-6, IL-8, IL-10, and IL-12), MIP-1, and/or MCP-1. As another example, certain IRM compounds can inhibit production and secretion of certain TH-2 cytokines, such as IL-4 and IL-5. Additionally, some IRM compounds are said to suppress IL-1 and TNF (U.S. Pat. No. 6,518,265).

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; and U.S. Patent Publication Nos. 2004/0091491; 2004/0147543; and 2004/0176367.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

The present invention provides immunomodualtory combinations useful for treating various types of disorders, such as, for example, cancers, infectious disorders, or a T_{H}2-mediated disorder.

IRM compounds suitable for use in the invention include compound containing a 2-aminopyridine fused to a five membered nitrogen-containing heterocyclic ring, or a 4-aminopyrimidine fused to a five membered nitrogen-containing heterocyclic ring. Suitable IRM compounds also may include the purine derivatives, imidazoquinoline amide derivatives, benzimidazole derivatives, adenine derivatives, and oligonucleotide sequences described above.

Certain IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In other alternative embodiments, the IRM compound may be an imidazonaphthyridine amine or a tetrahydroimidazonaphthyridine amine.

In yet other alternative embodiments, the IRM compound may be a sulfonamide substituted or ether substituted imidazoquinoline amine, tetrahydroimidazoquinoline amine, or imidazopyridine amine. Such compounds include, for example, sulfonamide substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, sulfonamido substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines.

Unless otherwise indicated, reference to a compound (whether an IRM compound, an antibody, an antigen, etc.) can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

Antibodies useful in the invention include monoclonal antibodies, polyclonal antibodies, murine antibodies, human antibodies, chimeric murine-human antibodies, and the like. In some embodiments, antibody fragments can be used provided such fragments possess both an Fc and at least one Fab portion.

In some embodiments, the IRM compound is administered at the same time as the antibody, while in other embodiments, it is administered prior to following antibody administration. If delivered prior to the administration of the antibody, the IRM compound can be administered 1, 2, 3, 4, 5, 6, 7, or more days prior to the administration of antibody. If administered after the administration of the antibody, the IRM compound can be administered 1, 2, 3, 4, 5, 6, 7, or more days after the administration of the antibody. In some preferred embodiments, the IRM compound is administered within 48 hours, within 36 hours, within 24 hours, within 12 hours, within 6 hours, or within 4 hours of antibody administration, regardless of whether the antibody is administered prior to or following the IRM compound.

Therapeutic antibodies useful in the invention may be specific for microbial antigens (e.g., bacterial, viral, parasitic or fungal antigens), cancer or tumor-associated antigens and self antigens. Preferred antibodies are those that recognize and bind to antigens present on or in a cell. Examples of suitable antibodies include but are not limited to Rituxan® (rituximab, anti-CD20 antibody), Herceptin (trastuzumab), Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART ID10Ab, SMART ABL 364 Ab, CC49 (mAb B72.3), ImmuRAIT-CEA, anti-IL-4 antibody, an anti-IL-5 antibody, an anti-IL-9 antibody, an anti-Ig antibody, an anti-IgE antibody, serum-derived hepatitis B antibodies, recombinant hepatitis B antibodies, and the like.

Other antibodies similarly useful for the invention include alemtuzumab (B cell chronic lymphocytic leukemia), gemtuzumab ozogamicin (CD33+ acute myeloid leukemia), hP67.6 (CD33+ acute myeloid leukemia), infliximab (inflammatory bowel disease and rheumatoid arthritis), etanercept (rheumatoid arthritis), tositumomab, MDX-210, oregovomab, anti-EGF receptor mAb, MDX-447, anti-tissue factor protein (TF), (Sunol); ior-c5, c5, edrecolomab, ibritumomab tiuxetan, anti-idiotypic mAb mimic of ganglioside GD3 epitope, anti-HLA-Dr10 mAb, anti-CD33 humanized mAb, anti-CD52 humAb, anti-CD1 mAb (ior t6), MDX-22, celogovab, anti-17-1A mAb, bevacizumab, daclizumab, anti-TAG-72 (MDX-220), anti-idiotypic mAb mimic of high molecular weight proteoglycan (I-Mel-1), anti-idiotypic mAb mimic of high molecular weight proteoglycan (I-Mel-2), anti-CEA Ab, hmAbH11, anti-DNA or DNA-associated proteins (histones) mAb, Gliomab-H mAb, GNI-250 mAb, anti-CD22, CMA 676), anti-idiotypic human mAb to GD2 ganglioside, ior egf/r3, anti-ior c2 glycoprotein mAb, ior c5, anti-FLK-2/FLT-3 mAb, anti-GD-2 bispecific mAb, antinuclear autoantibodies, anti-HLA-DR Ab, anti-CEA mAb, palivizumab, bevacizumab, alemtuzumab, BLyS-mAb, anti-VEGF2, anti-Trail receptor; B3 mAb, mAb BR96, breast cancer; and Abx-Cb1 mAb.

Suitable antibodies also include the following, all of which are commercially available:

Apoptosis Antibodies: BAX Antibodies: Anti-Human Bax Antibodies (Monoclonal), Anti-Human Bax Antibodies (Polyclonal), Anti-Murine Bax Antibodies (Monoclonal), Anti-Murine Bax Antibodies (Polyclonal); Fas/Fas Ligand Antibodies: Anti-HumanFas/Fas Ligand Antibodies, Anti-Murine Fas/Fas Ligand Antibodies Granzyme Antibodies Granzyme B Antibodies; BCL Antibodies: Anti Cytochrome C Antibodies, Anti-Human BCL Antibodies (Monoclonal), Anti-Human bcl Antibodies (Polyclonal), Anti-Murine bcl Antibodies (Monoclonal), Anti-Murine bcl Antibodies (Polyclonal);
Miscellaneous Apoptosis Antibodies: Anti TRADD, TRAIL, TRAFF, DR3 Antibodies Anti-Human Fas/Fas Ligand Antibodies Anti-Murine Fas/Fas Ligand Antibodies;
Miscellaneous Apoptosis Related Antibodies: BIM Antibodies: Anti Human, Murine bim Antibodies (Polyclonal), Anti-Human, Murine bim Antibodies (Monoclonal);
PARP Antibodies: Anti-Human PARP Antibodies (Monoclonal), Anti-Human PARP Antibodies (Polyclonal), Anti-Murine PARP Antibodies;
Caspase Antibodies: Anti-Human Caspase Antibodies (Monoclonal), Anti-Murine Caspase Antibodies;
Anti-CD Antibodies: Anti-CD29, PL18-5 PanVera, Anti-CD29, PL4-3 PanVera, Anti-CD41a, PT25-2 PanVera, Anti-CD42b, PL52-4 PanVera, Anti-CD42b, GUR20-5 PanVera, Anti-CD42b, WGA-3 PanVeraAnti-CD43, 1D4 PanVera, Anti-CD46, MCP75-6 PanVera, Anti-CD61, PL11-7 PanVera, Anti-CD61, PL8-5 PanVera, Anti-CD62/P-slctn, PL7-6 PanVera, Anti-CD62/P-slctn, WGA-1 PanVera, Anti-CD154, 5F3 PanVera;
Human Chemokine Antibodies: Human CNTF Antibodies, Human Eotaxin Antibodies, Human Epithelial Neutrophil Activating Peptide-78, Human Exodus Antibodies, Human GRO Antibodies, Human HCC-1 Antibodies, Human I-309 Antibodies, Human IP-10Antibodies, Human I-TAC Antibodies, Human LIF Antibodies, Human Liver-Expressed Chemokine Antibodies, Human Lymphotaxin Antibodies, Human MCP Antibodies, Human MIP Antibodies, Human Monokine Induced by IFN-γ Antibodies, Human NAP-2 Antibodies, Human NP-1 Antibodies, Human Platelet Factor-4 Antibodies, Human RANTES Antibodies, Human SDF Antibodies, Human TECK Antibodies;
Murine Chemokine Antibodies: Human B-Cell Attracting Murine Chemokine Antibodies, Chemokine-1 Antibodies, Murine Eotaxin Antibodies, Murine Exodus Antibodies, Murine GCP-2 Antibodies, Murine KC Antibodies, Murine MCP Antibodies, Murine MIP Antibodies, Murine RANTES Antibodies, Rat Chemokine Antibodies, Rat Chemokine Antibodies, Rat CNTF Antibodies, Rat GRO Antibodies, Rat MCP Antibodies, Rat MIP Antibodies, Rat RANTES Antibodies;
Cytokine/Cytokine Receptor Antibodies: Human Biotinylated Cytokine/Cytokine Receptor Antibodies, Human IFN Antibodies, Human IL Antibodies, Human Leptin Antibodies, Human Oncostatin Antibodies, Human TNF Antibodies, Human TNF Receptor Family Antibodies, Murine Biotinylated Cytokine/Cytokine Receptor Antibodies, Murine IFN Antibodies, Murine IL Antibodies, Murine TNF Antibodies, Murine TNF Receptor Antibodies;
Rat Cytokine/Cytokine Receptor Antibodies: Rat Biotinylated Cytokine/Cytokine Receptor Antibodies, Rat IFN Antibodies, Rat IL Antibodies, Rat TNF Antibodies;
ECM Antibodies: Collagen/Procollagen, Laminin, Collagen (Human), Laminin (Human), Procollagen (Human), Vitronectin/Vitronectin Receptor, Vitronectin (Human), Vitronectin Receptor (Human), Fibronectin/Fibronectin Receptor, Fibronectin (Human), Fibronectin Receptor (Human);
Growth Factor Antibodies: Human Growth Factor Antibodies, Murine Growth Factor Antibodies, Porcine Growth Factor Antibodies;
Miscellaneous Antibodies: Baculovirus Antibodies, Cadherin Antibodies, Complement Antibodies, C1q Antibodies, VonWillebrand Factor Antibodies, Cre Antibodies, HIV Antibodies, Influenza Antibodies, Human Leptin Antibodies, Murine LeptinAntibodies, Murine CTLA-4 Antibodies, P450 Antibodies, RNA Polymerase Antibodies;
Neurobio Antibodies: Amyloid Antibodies, GFAP Antibodies, Human NGF Antibodies, Human NT-3 Antibodies, Human NT-4 Antibodies.

Additional antibodies suitable for use in the invention include, for example, antibodies listed in references such as the MSRS Catalog of Primary Antibodies and Linscott's Directory.

The IRM compounds can also be used with normal and hyper-immune globulin therapy. Normal immune globulin therapy utilizes an antibody product that is prepared from the serum of normal blood donors and pooled. This pooled product contains low titers of antibody to a wide range of antigens such as those of infectious pathogens (e.g., bacteria, viruses such as hepatitis A, parvovirus, enterovirus, fungi and parasites). Hyper-immune globulin therapy utilizes antibodies that are prepared from the serum of individuals who have high titers of an antibody to a particular antigen. Examples of hyper-immune globulins include zoster immune globulin (useful for the prevention of varicella in immunocompromised children and neonates), human rabies immunoglobulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

The invention is further based, in part, on the surprising discovery that administration of an IRM compound and a therapeutic agent has unexpected benefit over the administration of either compound alone. Of particular importance is the use of immunostimulatory nucleic acids, C8-substituted guanosines, antigens, and disorder specific medicaments as therapeutic agents. In one embodiment, compositions comprising IRM compounds, immunostimulatory nucleic acids, antigen and a polymer rich in arginine (e.g., polyarginine), and optionally C8-substituted guanosine are used in the immunomodulatory methods of the invention.

The IRM compounds are also useful for redirecting an immune response to a T_{H}1 immune response. Redirection of an immune response to a T_{H}1 immune response can be assessed by measuring the levels of cytokines produced in response to the IRM compound (e.g., by inducing monocytic cells and other cells to produce T_{H}1 cytokines, including IL-12, IFN-α and GM-CSF). The redirection or rebalance of the immune response to a T_{H}1 response is particularly useful for the treatment or prevention of asthma. For instance, an effective amount for treating asthma can be that amount useful for redirecting a T_{H}2 type of immune response that is associated with asthma to a T_{H}1 type of response. T_{H}2 cytokines, especially IL-4 and IL-5, are elevated in the airways of asthmatic subjects. These cytokines promote important aspects of the asthmatic inflammatory response including, for example, IgE isotype switching, eosinophil chemotaxis and activation, and mast cell growth. T_{H}1 cytokines, especially IFN-α and IL-12, can suppress the formation of T_{H}2 clones and production of T_{H}2 cytokines. The immunomodulatory combinations of the invention can cause an increase in T_{H}1 cytokines, which helps to rebalance the immune system, preventing or reducing the adverse effects associated with a predominately T_{H}2 immune response. The redirection of a T_{H}2 to a T_{H}1 immune response may result in a balanced expression of T_{H}1 and T_{H}2 cytokines or it may result in the induction of more T_{H}1 cytokines than T_{H}2 cytokines.

The invention also includes a method for inducing antigen non-specific innate immune activation and broad spectrum resistance to infectious challenge using the IRM compounds. The term antigen non-specific innate immune activation as used herein refers to the activation of immune cells other than B cells and can include the activation of, for example, NK cells, T cells, other immune cells that can respond in an antigen independent fashion, or some combination of these cells. A broad spectrum resistance to infectious challenge is induced because the immune cells are in active form and are primed to respond to any invading compound or microorganism. The cells do not have to be specifically primed against a particular antigen. This may be particularly useful for providing immunological protection against an unknown or, alternatively, multiple infectious agents. Methods and composition useful in this regard are described, for example, in co-pending U.S. patent application Ser. No. 10/911,800, filed Aug. 5, 2004.

The stimulation index of a particular IRM compound can be tested in various immune cell assays. Preferably, the stimulation index of the IRM compound with regard to B cell proliferation is at least about 5, preferably at least about 10, more preferably at least about 15 and most preferably at least about 20 as determined by incorporation of ³H uridine in a murine B cell culture, which has been contacted with 20 μM of nucleic acid for 20 hours at 37° C. and has been pulsed with 1 μCi of ³H-uridine; and harvested and counted 4 hours later as described in detail in U.S. Pat. Nos. 6,207,646B1 and 6,239, 116B1 with respect to immunostimulatory nucleic acids. For use in vivo, for example, it is important that the IRM compounds be capable of effectively inducing an immune response, such as, for example, antibody production.

Currently, some treatment protocols for certain disorders (e.g., cancer) call for the use of IFN-α. In one embodiment, the methods of the invention use IRM compounds as a replacement to the use of IFN-α therapy in the treatment of certain disorders. IRM compounds can be used to generate IFN-α endogenously. In yet other embodiments, the IRM compounds may be administered along with IFN-α. In some embodiments, the targeting agent of the invention or a disorder-specific medicament can also be administered to the subject along with the IRM compound and IFN-α.

The invention embraces the administration of C8-substituted guanosines either in place of or along with the IRM compounds in the methods of the invention. C8-substituted guanosines are known to activate both natural killer (NK) cells and macrophages. Guanine ribonucleotides substituted at the C8 position with either a bromine or a thiol group are B cell mitogens and may act as B cell differentiation factors. These compounds have been reported to reduce the IL-2 requirement for NK cell activation. NK and LAK augmenting activities of C8-substituted guanosines appear to be due to their induction of IFN. Examples of C8-substituted guanosines include but are not limited to 8-mercaptoguanosine, 8-bromoguanosine, 8-methylguanosine, 8-oxo-7,8-dihydroguanosine, C8-arylamino-2′-deoxyguanosine, C8-propynyl-guanosine, C8- and N7-substituted guanine ribonucleosides such as 7-allyl-8-oxoguanosine (loxoribine) and 7-methyl-8-oxoguanosine, 8-aminoguanosine, 8-hydroxy-2′-deoxyguanosine, and 8-hydroxyguanosine. 8-mercaptoguanosine and 8-bromoguanosine also can substitute for the cytokine requirement for the generation of MHC restricted CTL, augment murine NK activity, and synergize with IL-2 in inducing murine LAK generation. In some important embodiments of the invention, C8-substituted guanosines can be used together with or in place of IRM compounds for the purpose of inducing or enhancing an immune response that includes ADCC.

Certain methods and compositions of the invention comprise the administration or addition of polyarginine. As used herein, polyarginine is a homogenous polymer of arginine monomers. Polyarginine may be of varying length, and may have a peptide backbone but is not so limited. In other embodiments, a polymer rich in arginine can also be used in place of the homogenous polymer of arginine. A polymer rich in arginine can be a polymer that has at least 2 contiguous arginines, at least 3 contiguous arginines, at least 4 contiguous arginines, and at least 5 contiguous arginines, or alternatively it may be a polymer in which at least 20%, at least 30%, at least 40%, at least 50%, at least 60%, at least 70%, at least 80%, or at least 90% of its monomers are arginine residues. It is to be understood, accordingly, that polyarginine is also a polymer rich in arginine. Because of the positive charge of arginine, polymers rich in arginine (including polyarginine) serve to neutralize the negative charge associated with some IRM compounds and the immunostimulatory nucleic acids.

An “immunostimulatory nucleic acid” as used herein is any nucleic acid containing an immunostimulatory motif or backbone that induces an immune response. The immune response may be characterized as, but is not limited to, a T_{H}1-type immune response or a T_{H}2-type immune response. Such immune responses are defined by cytokine and antibody production profiles which are elicited by the activated immune cells. In one preferred embodiment, pan activating immunostimulatory nucleic acids such as #2006 (TCG TCG TTT TGT CGT TTT GTC GTT) are used in combination with the IRM compounds in the methods of the invention.

Helper (CD4+) T cells orchestrate the immune response of mammals through production of soluble factors that act on other immune system cells, including other T cells. Helper CD4+, and in some instances also CD8+, T cells are characterized as T_{H}1 and T_{H}2 cells (and Tc1 and Tc2 cells if CD8+) in both murine and human systems, depending on their cytokine production profiles (Romagnani, 1991, Immunol. Today 12: 256-257; Mosmann, 1989, Annu. Rev. Immunol., 7: 145-173). T_{H}1 cells produce IL-2, IL-12, TNF-α and IFN-γ and they are responsible primarily for cell-mediated immunity such as delayed type hypersensitivity. The cytokines that are induced by administration of immunostimulatory nucleic acids are predominantly of the T_{H}1 class. The types of antibodies associated with a T_{H}1 response are generally more protective because they have high neutralization and opsonization capabilities. T_{H}2 cells produce IL-4, IL-5, IL-6, IL-9, IL-10 and IL-13 and are primarily involved in providing optimal help for humoral immune responses such as IgE and IgG4 antibody isotype switching (Mosmann, 1989, Annu. Rev. Immunol, 7: 145-173). T_{H}2 responses involve predominantly antibodies that have less protective effects against infection.

The terms “nucleic acid” and “oligonucleotide” are used interchangeably to mean multiple nucleotides (i.e., molecules comprising a sugar (e.g., ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acid molecules can be obtained from existing nucleic acid sources (e.g., genomic or cDNA), but are preferably synthetic (e.g. produced by nucleic acid synthesis). Nucleic acids suitable for use in the invention are described for example in United States Patent Publication No. U.S. 2003/0139364.

Immunostimulatory nucleic acids may possess immunostimulatory motifs such as CpG, poly-G, poly-T, TG, methylated CpG, CpI, and T-rich motifs. In some embodiments of the invention, any nucleic acid, regardless of whether it possesses an identifiable motif, can be used in the combination therapy to modulate an immune response. Immunostimulatory backbones include, but are not limited to, phosphate modified backbones, such as phosphorothioate backbones. Immunostimulatory nucleic acids have been described extensively in the prior art and a brief summary of these nucleic acids is presented below.

In some embodiments, a CpG immunostimulatory nucleic acid is used in the methods of the invention. A CpG immunostimulatory nucleic acid is a nucleic acid that contains a CG dinucleotide, the C residue of which is unmethylated. The effects of CpG nucleic acids on immune modulation have been described extensively in U.S. patent such as, for example, U.S. Pat. No. 6,194,388 B1; U.S. Pat. No. 6,207,646 B1; U.S. Pat. No. 6,239,116 B1; and U.S. Pat. No. 6,218,371 B1; and published international patent applications, such as PCT/US98/03678, PCT/US98/10408, PCT/US98/04703, and PCT/US99/09863.

The terms CpG nucleic acid or CpG oligonucleotide as used herein refer to an immunostimulatory CpG nucleic acid unless otherwise indicated. The entire immunostimulatory nucleic acid can be unmethylated or portions may be unmethylated but at least the C of the 5′ CG 3′ must be unmethylated.

The CpG nucleic acid sequences of the invention include those broadly described above as well as disclosed in issued U.S. Pat. Nos. 6,207,646 B1 and 6,239,116 B1.

The therapeutic agents described herein including IRM compounds, antigens, immunostimulatory nucleic acids, antibodies, C8-substituted guanosines, as well as the polymers rich in arginine can be physically combined without the need for covalent bonding between their substituents when used in the methods of the invention. Alternatively, they may also be conjugated in various combinations either directly or indirectly using linking molecules, as described below.

Examples of suitable linking molecules that can be used include bifunctional crosslinker molecules. The bifunctional crosslinker molecules may be homobifunctional or heterobifunctional, depending upon the nature of the molecules to be conjugated. Homobifunctional crosslinkers have two identical reactive groups. Heterobifunctional crosslinkers are defined as having two different reactive groups that allow for sequential conjugation reaction. Various types of commercially available crosslinkers are reactive with one or more of the following groups: primary amines, secondary amines, sulphydryls, carboxyls, carbonyls and carbohydrates. Examples of amine-specific crosslinkers are bis(sulfosuccinimidyl) suberate, bis[2-(succinimidooxycarbonyloxy)ethyl]sulfone, disuccinimidyl suberate, disuccinimidyl tartarate, dimethyl adipimate.2 HCl, dimethyl pimelimidate.2 HCl, dimethyl suberimidate.2 HCl, and ethylene glycolbis-[succinimidyl-[succinate]]. Crosslinkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido) butyl]-3′-[2′-pyridyldithio]propionamide. Crosslinkers preferentially reactive with carbohydrates include azidobenzoyl hydrazine. Crosslinkers preferentially reactive with carboxyl groups include 4-[p-azidosalicylamido]butylamine. Heterobifunctional crosslinkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional crosslinkers that react with carboxyl and amine groups include 1-ethyl-3-[3-dimethylaminopropyl]carbodiimide hydrochloride. Heterobifunctional crosslinkers that react with carbohydrates and sulfhydryls include 4-[N-maleimidomethyl]-cyclohexane-1-carboxylhydrazide. 2 HCl, 4-(4-N-maleimidophenyl)-butyric acid hydrazide.2 HCl, and 3-[2-pyridyldithio]propionyl hydrazide. The crosslinkers are bis-[α-4-azidosalicylamido)ethyl]disulfide and glutaraldehyde. Amine or thiol groups may be added at any nucleotide of a synthetic nucleic acid molecule so as to provide a point of attachment for a bifunctional crosslinker molecule. The nucleic acid molecule may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

Additional methods of crosslinking, as well as non-covalently pairing, IRM compounds and therapeutic agents are described, for example, in U.S. Patent Publication No. U.S. 2004/0091491.

The IRM compounds together with the other agents described herein are useful in some aspects of the invention in the prophylaxis and treatment of subjects having or at risk of developing (i.e., at risk of having) a disorder. Generally, the disorders to be prevented and/or treated by the methods provided herein are those that would benefit from a stimulated immune response. In important embodiments, the disorders targeted by the methods and compositions of the invention include cancer, infectious disease, and asthma and allergy. The disorder may also be warts.

The invention intends to treat subjects who are at risk of developing particular disorders (e.g., infectious disease, cancer, asthma, allergy and disorders characterized by warts), as well as subjects that have such disorders. As used herein, the term treat, treated, or treating when used with respect to one of the disorders described herein refers to a prophylactic treatment which decreases the likelihood that the subject will develop the disorder as well as a treatment after the subject has developed the disorder, e.g., reduce or eliminate the disorder or prevent it from becoming worse. Subjects at risk are defined as those who have a higher than normal risk of developing the disorder. The normal risk is generally the risk of a population of normal individuals who do not have the disorder and are not at risk of developing it.

Thus, in prophylactic methods of the invention, the subjects to be treated include those that are at risk of developing an infectious disease, those at risk of developing cancer, and those at risk of developing asthma or allergy. A subject at risk of developing a disorder generally refers to a subject that has a greater likelihood of having the disorder than the population on average.

A subject shall mean a human or animal including but not limited to a dog, cat, horse, cow, pig, sheep, goat, chicken, rodent e.g., rats and mice, primate, e.g., monkey, and fish or aquaculture species such as fin fish (e.g., salmon) and shellfish (e.g., shrimp and scallops). Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. Although many of the embodiments described herein relate to human disorders, the invention is also useful for treating other nonhuman vertebrates. Nonhuman vertebrates are also capable of being treated with the IRM compounds disclosed herein.

An “infectious disease” as used herein, refers to a disorder arising from the invasion of a host, superficially, locally, or systemically, by an infectious organism. Infectious organisms include bacteria, viruses, fungi, and parasites. Accordingly, “infectious disease” includes bacterial infections, viral infections, fungal infections and parasitic infections.

Bacteria are unicellular organisms that multiply asexually by binary fission. They are classified and named based on their morphology, staining reactions, nutrition and metabolic requirements, antigenic structure, chemical composition, and genetic homology. Bacteria can be classified into three groups based on their morphological forms, spherical (coccus), straight-rod (bacillus) and curved or spiral rod (vibrio, campylobacter, spirillum, and spirochaete). Bacteria are also more commonly characterized based on their staining reactions into two classes of organisms, Gram-positive and Gram-negative. Gram refers to the method of staining which is commonly performed in microbiology labs. Gram-positive organisms retain the stain following the staining procedure and appear a deep violet color. Gram-negative organisms do not retain the stain but take up the counter-stain and thus appear pink.

Viruses are small infectious agents that generally contain a nucleic acid core and a protein coat, but are not independently living organisms. Viruses can also take the form of infectious nucleic acids lacking a protein. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and other are RNA-containing viruses.

Viruses include, but are not limited to, enteroviruses (including, but not limited to, viruses that the family picornaviridae, such as polio virus, coxsackie virus, echo virus), rotaviruses, adenovirus, hepatitis.

Infectious viruses of both human and non-human vertebrates, include retroviruses, RNA viruses and DNA viruses. This group of retroviruses includes both simple retroviruses and complex retroviruses. The simple retroviruses include the subgroups of B-type retroviruses, C-type retroviruses and D-type retroviruses.

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as amatatoxin and phallotoxin produced by poisonous mushrooms and aflotoxins, produced by aspergillus species, not all fungi cause infectious disease.

Infectious fungi can cause systemic or superficial infections. Primary systemic infection can occur in normal healthy subjects and opportunistic infections, are most frequently found in immuno-compromised subjects. The most common fungal agents causing primary systemic infection include blastomyces, coccidioides, and histoplasma. Common fungi causing opportunistic infection in immunocompromised or immunosuppressed subjects include, but are not limited to, candida albicans, cryptococcus neoformans, and various aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi.

Superficial fungal infections involve growth of fungi on an external surface without invasion of internal tissues. Typical superficial fungal infections include cutaneous fungal infections involving skin, hair, or nails.

Diseases associated with fungal infection include aspergillosis, blastomycosis, camdidiais, chromoblastomycosis, coccidioidomycosis, cryptococcosis, fungal eye infections, fungal hair, nail, and skin infections, histoplasmosis, lobomycosis, mycetoma, otomycosis, paracoccidioidomycosis, penicilliosis, marneffeii, phaeohyphomycosis, rhinosporidioisis, sporotrichosis, and zygomycosis.

Parasites are organisms that depend upon other organisms in order to survive and thus must enter, or infect, another organism to continue their life cycle. The infected organism, i.e., the host, provides both nutrition and habitat to the parasite. Although in its broadest sense the term parasite can include all infectious agents (i.e., bacteria, viruses, fungi, protozoa and helminths), generally speaking, the term is used to refer solely to protozoa, helminths, and ectoparasitic arthropods (e.g., ticks, mites, etc.). Protozoa are single celled organisms that can replicate both intracellularly and extracellularly, particularly in the blood, intestinal tract or the extracellular matrix of tissues. Helminths are multicellular organisms that almost always are extracellular (the exception being Trichinella spp.). Helminths normally require exit from a primary host and transmission into a secondary host in order to replicate. In contrast to these aforementioned classes, ectoparasitic arthropods form a parasitic relationship with the external surface of the host body.

Parasites include intracellular parasites and obligate intracellular parasites. Examples of parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica, Trypanosoma gambiense, Trypanosmoma rhodesiense and Schistosoma mansoni.

Other medically relevant microorganisms have been described extensively in the literature, e.g., see C. G. A Thomas, Medical Microbiology, Bailliere Tindall, Great Britain 1983. Each of the foregoing lists is illustrative, and is not intended to be limiting.

In some aspects, the invention also intends to treat diseases in which prions are implicated in disease progression such as for example bovine spongiform encephalopathy (i.e., mad cow disease) or scrapie infection in animals, or Creutzfeldt-Jakob disease in humans.

In some embodiments, the methods of the invention are intended to treat or prevent infection such as small pox or anthrax infections.

A subject having an infectious disease is a subject that has been exposed to an infectious organism and has acute or chronic detectable levels of the organism in the body. Exposure to the infectious organism generally occurs with the external surface of the subject, e.g., skin or mucosal membranes and/or refers to the penetration of the external surface of the subject by the infectious organism.

A subject at risk of developing an infectious disease is a subject who has a higher than normal risk of exposure to an infection causing pathogen. For instance, a subject at risk may be a subject who is planning to travel to an area where a particular type of infectious agent is found or it may be a subject who through lifestyle or medical procedures is exposed to bodily fluids which may contain infectious organisms or directly to the organism or a subject living in an area where an infectious organism has been identified. Subjects at risk of developing an infectious disease also include general populations to which a medical agency recommends vaccination against a particular infectious organism.

A subject at risk of developing an infectious disease includes those subjects that have a general risk of exposure to a microorganism, e.g., influenza, but that don't have the active disease during the treatment of the invention as well as subjects that are considered to be at specific risk of developing an infectious disease because of medical or environmental factors, that expose them to a particular microorganism.

Cancer is a disease that involves the uncontrolled growth (i.e., division) of cells. Some of the known mechanisms which contribute to the uncontrolled proliferation of cancer cells include growth factor independence, failure to detect genomic mutation, and inappropriate cell signaling. The ability of cancer cells to ignore normal growth controls may result in an increased rate of proliferation. Although the causes of cancer have not been firmly established, there are some factors known to contribute, or at least predispose a subject, to cancer. Such factors include particular genetic mutations (e.g., BRCA gene mutation for breast cancer, APC for colon cancer), exposure to suspected cancer-causing agents, or carcinogens (e.g., asbestos, UV radiation) and familial disposition for particular cancers such as breast cancer.

The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

A subject having a cancer is a subject that has detectable cancerous cells.

A subject at risk of developing a cancer is one who has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

An “allergy” refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions atopic dermatitis; anaphylaxis; drug allergy; angioedema; and allergic conjunctivitis. Allergic diseases in dogs include but are not limited to seasonal dermatitis; perennial dermatitis; rhinitis: conjunctivitis; allergic asthma; and drug reactions. Allergic diseases in cats include but are not limited to dermatitis and respiratory disorders, and food allergens. Allergic diseases in horses include but are not limited to respiratory disorders such as “heaves” and dermatitis. Allergic diseases in non-human primates include but are not limited to allergic asthma and allergic dermatitis.

Allergy is a disease associated with the production of antibodies from a particular class of immunoglobulin, IgE, against allergens. The development of an IgE-mediated response to common aeroallergens is also a factor that indicates predisposition towards the development of asthma. If an allergen encounters a specific IgE that is bound to an Fc IgE receptor on the surface of a basophil (circulating in the blood) or mast cell (dispersed throughout solid tissue), the cell becomes activated, resulting in the production and release of mediators such as histamine, scrotonin, and lipid mediators. Allergic diseases include but are not limited to rhinitis (hay fever) asthma, urticaria and atopic dermatitis.

A subject having an allergy is a subject that is currently experiencing or has previously experienced an allergic reaction in response to an allergen.

A subject at risk of developing an allergy or asthma is a subject that has been identified as having an allergy or asthma in the past but who is not currently experiencing the active disease as well as a subject that is considered to beat risk of developing asthma or allergy because of genetic or environmental factors. A subject at risk of developing allergy or asthma can also include a subject who has any risk of exposure to an allergen or a risk of developing asthma, i.e. someone who has suffered from an asthmatic attack previously or has a predisposition to asthmatic attacks. For instance, a subject at risk may be a subject who is planning to travel to an area where a particular type of allergen or asthmatic initiator is found or it may even be any subject living in an area where an allergen has been identified. If the subject develops allergic responses to a particular antigen and the subject may be exposed to the antigen, i.e., during pollen season, then that subject is at risk of exposure to the antigen.

Allergic diseases may be treated by injecting small doses of antigen followed by subsequent increasing dosage of antigen. It is believed that this procedure induces tolerization to the allergen to prevent further allergic reactions. These methods, however, can take several years to be effective and are associated with the risk of side effects such as anaphylactic shock. The methods of the invention avoid these problems.

Allergies are generally caused by IgE antibody generation against harmless allergens. The cytokines that are induced by systemic or mucosal administration of IRM compounds are predominantly of a class called T_{H}1 (examples are IL-12, IFN-α and IFN-γ) and these induce both humoral and cellular immune responses. The types of antibodies associated with a T_{H}1 response are generally more protective because they have high neutralization and opsonization capabilities. The other major type of immune response, which is associated with the production of IL-4, IL-5 and IL-10 cytokines, is termed a T_{H}2 immune response. T_{H}2 responses involve predominately antibodies and these have less protective effect against infection and some T_{H}2 isotypes (e.g., IgE) are associated with allergy. In general, it appears that allergic diseases are mediated by T_{H}2 type immune responses while T_{H}1 responses provide the best protection against infection, although excessive T_{H}1 responses are associated with autoimmune disease. Based on the ability of the IRM compounds to shift the immune response in a subject to a T_{H}1 response (which is protective against allergic reactions), an effective dose for inducing an immune response of a IRM compound can be administered to a subject to treat or prevent an allergy.

The generic name for molecules that cause an allergic reaction is allergen. There are numerous species of allergens. The allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

The symptoms of the allergic reaction vary, depending on the location within the body where the IgE reacts with the antigen. If the reaction occurs along the respiratory epithelium the symptoms are sneezing, coughing and asthmatic reactions. If the interaction occurs in the digestive tract, as in the case of food allergies, abdominal pain and diarrhea are common. Systematic reactions, for example following a bee sting, can be severe and often life threatening.

Delayed type hypersensitivity, also known as type IV allergy reaction is an allergic reaction characterized by a delay period of at least 12 hours from invasion of the antigen into the allergic subject until appearance of the inflammatory or immune reaction. The T lymphocytes (sensitized T lymphocytes) of individuals in an allergic condition react with the antigen, triggering the T lymphocytes to release lymphokines (macrophage migration inhibitory factor (MIF), macrophage activating factor (MAF), mitogenic factor (MF), skin-reactive factor (SRF), chemotactic factor, neovascularization-accelerating factor, etc.), which function as inflammation mediators, and the biological activity of these lymphokines, together with the direct and indirect effects of locally appearing lymphocytes and other inflammatory immune cells, give rise to the type IV allergy reaction. Delayed allergy reactions include tuberculin type reaction, homograft rejection reaction, cell-dependent type protective reaction, contact dermatitis hypersensitivity reaction, and the like, which are known to be most strongly suppressed by steroidal agents. Consequently, steroidal agents are effective against diseases that are caused by delayed allergy reactions. Long-term use of steroidal agents at concentrations currently being used can, however, lead to the serious side-effect known as steroid dependence. The methods of the invention solve some of these problems, by providing for lower and fewer doses to be administered.

Immediate hypersensitivity (or anaphylactic response) is a form of allergic reaction that develops very quickly, i.e., within seconds or minutes of exposure of the patient to the causative allergen, and it is mediated by IgE antibodies made by B lymphocytes. In nonallergic patients, there is no IgE antibody of clinical relevance; but, in a person suffering with allergic diseases, IgE antibody mediates immediate hypersensitivity by sensitizing mast cells which are abundant in the skin, lymphoid organs, in the membranes of the eye, nose and mouth, and in the respiratory tract and intestines.

Mast cells have surface receptors for IgE, and the IgE antibodies in allergy-suffering patients become bound to them. As discussed briefly above, when the bound IgE is subsequently contacted by the appropriate allergen, the mast cell is caused to degranulate and to release various substances called bioactive mediators, such as histamine, into the surrounding tissue. It is the biologic activity of these substances which is responsible for the clinical symptoms typical of immediate hypersensitivity; namely, contraction of smooth muscle in the airways or the intestine, the dilation of small blood vessels and the increase in their permeability to water and plasma proteins, the secretion of thick sticky mucus, and in the skin, redness, swelling and the stimulation of nerve endings that results in itching or pain.

The IRM compounds have significant therapeutic utility in the treatment of allergic and non-allergic conditions such as asthma, particularly when used in combination with other therapeutic agents (e.g., those used to regulate levels of proinflammatory cytokines). T_{H}2 cytokines, especially IL-4 and IL-S are elevated in the airways of asthmatic subjects. These cytokines promote important aspects of the asthmatic inflammatory response, including IgE isotope switching, eosinophil chemotaxis and activation and mast cell growth. T_{H}1 cytokines, especially IFN-γ and IL-12, can suppress the formation of T_{H}2 clones and production of T_{H}2 cytokines. Asthma refers to a disorder of the respiratory system characterized by inflammation, narrowing of the airways and increased reactivity of the airways to inhaled agents. Asthma is frequently, although not exclusively associated with atopic or allergic symptoms. In some of the preceding aspects of the invention related to asthma and allergy, the IRM compounds of the invention are not administered directly to the lungs of the subject. Additional methods of treating T_{H}2-mediated disorders are described, for example, in U.S. Pat. No. 6,696,076 and co-pending U.S. patent application Ser. No. 10/738,853, filed Dec. 17, 2003.

Symptoms of asthma include recurrent episodes of wheezing, breathlessness, and chest tightness, and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, subbasement membrane fibrosis may occur, leading to persistent abnormalities in lung function.

Asthma may result from complex interactions among inflammatory cells, mediators, and other cells and tissues resident in the airway. Mast cells, eosinophils, epithelial cells, macrophage, and activated T-cells all play an important role in the inflammatory process associated with asthma (Djukanovic et al., Am. Rev. Respir. Dis; 142: 434-457, 1990). It is believed that these cells can influence airway function through secretion of preformed and newly synthesized mediators that can act directly or indirectly on the local tissue. It has also been recognized that subpopulations of T-lymphocytes (T_{H}2) play an important role in regulating allergic inflammation in the airway by releasing selective cytokines and establishing disease chronicity (Robinson, et al. N. Engl. J. Med.; 326: 298-304; 1992).

Asthma is a complex disorder that arises at different stages in development and can be classified based on the degree of symptoms of acute, subacute or chronic. An acute inflammatory response is associated with an early recruitment of cells into the airway. The subacute inflammatory response involves the recruitment of cells as well as the activation of resident cells causing a more persistent pattern of inflammation. Chronic inflammatory response is characterized by a persistent level of cell damage and an ongoing repair process, which may result in permanent abnormalities in the airway.

A “subject having asthma” is a subject that has a disorder of the respiratory system characterized by inflammation, narrowing of the airways and increased reactivity of the airways to inhaled agents. Asthma is frequently, although not exclusively associated with atopic or allergic symptoms. An “initiator” as used herein refers to a composition or environmental condition that triggers asthma. Initiators include, but are not limited to, allergens, cold temperatures, exercise, viral infections, and SO₂.

In another aspect the invention provides methods for treating or preventing a disorder in a hypo-responsive subject. As used herein, a hypo-responsive subject is one who has previously failed to respond to a treatment directed at treating or preventing the disorder or one who is at risk of not responding to such a treatment.

Other subjects who are hypo-responsive include those who are refractory to a disorder-specific medicament. As used herein, the term “refractory” means resistant or failure to yield to treatment. Such subjects may be those who never responded to the medicament (i.e., subjects who are non-responders), or alternatively, they may be those who at one time responded to the medicament, but have since that time have become refractory to it. In some embodiments, the subject is one who is refractory to a subset of medicaments. A subset of medicaments is at least one medicament. In some embodiments, a subset refers to 2, 3, 4, 5, 6, 7, 8, 9, or 10 medicaments.

In other embodiments, hypo-responsive subjects are elderly subjects, regardless of whether they have or have not previously responded to a treatment directed at treating or preventing the disorder. Elderly subjects, even those who have previously responded to such treatment, are considered to be at risk of not responding to a future administration of this treatment. Similarly, neonatal subjects are also considered to be at risk of not responding to treatment directed at treating or preventing the disorder. In important embodiments, the disorder is asthma or allergy.

In some aspects, the methods of the invention include exposing the subject to be treated with an antigen prior to, concurrently with, or subsequent to the administration of an IRM compound.

As used herein, the term “exposed to” refers to either the active step of contacting the subject with an antigen or the passive exposure of the subject to the antigen in vivo. Methods for the active exposure of a subject to an antigen are well-known in the art. In general, an antigen is administered directly to the subject by any means such as intravenous, intramuscular, oral, transdermal, mucosal, intranasal, intratracheal, or subcutaneous administration. The antigen can be administered systemically or locally. Methods for administering the antigen and the IRM compounds are described in more detail below.

A subject is passively exposed to an antigen if an antigen becomes available for exposure to the immune cells in the body. A subject may be passively exposed to an antigen, for instance, by entry of a foreign pathogen into the body or by the development of a tumor cell expressing a foreign antigen on its surface.

The methods in which a subject is passively exposed to an antigen can be particularly dependent on timing of administration of the IRM compounds. For instance, in a subject at risk of developing a cancer or an infectious disease or an allergic or asthmatic response, the subject may be administered the IRM compounds on a regular basis when that risk is greatest, i.e., during allergy season or after exposure to a cancer causing agent. Additionally the IRM compounds may be administered to travelers before they travel to areas where they may be at risk of exposure to infectious agents. Likewise the IRM compounds may be administered to those (military and/or civilian) at risk of exposure to biowarfare to induce a systemic or mucosal immune response to the antigen when and if the subject is exposed to it.

In some cases it is desirable to administer an antigen with the IRM compound and in other cases no antigen is delivered. An antigen is a molecule capable of provoking an immune response. The term antigen broadly includes any type of molecule that is recognized by a host system as being foreign. Antigens include but are not limited to microbial antigens, cancer antigens, and allergens.

Antigens include, but are not limited to, cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many antigens are protein or polypeptide in nature, as proteins and polypeptides are generally more antigenic than carbohydrates or fats.

The term substantially purified as used herein refers to a polypeptide preparation that is substantially free of other proteins, lipids, carbohydrates or other materials with which it is naturally associated. One skilled in the art can purify viral or bacterial polypeptides using standard techniques for protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the viral or bacterial polypeptide can also be determined by amino-terminal amino acid sequence analysis. Other types of antigens not encoded by a nucleic acid vector such as polysaccharides, small molecule, mimics etc are described above, and included within the invention.

A microbial antigen as used herein is an antigen of a microorganism and includes but is not limited to virus, bacteria, parasites, and fungi. Such antigens include the intact organism as well as natural isolates and fragments or derivatives thereof and also synthetic compounds that are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

Polypeptides of bacterial pathogens include but are not limited to an iron-regulated outer membrane protein, (IROMP), an outer membrane protein (OMP), and an A-protein of Aeromonis salmonicida which causes furunculosis, p57protein of Renibacterium salmoninarum which causes bacterial kidney disease (BKD), major surface associated antigen (msa), a surface expressed cytotoxin (mpr), a surface expressed hemolysin (ish), and a flagellar antigen of Yersiniosis; an extracellular protein (ECP), an iron-regulated outer membrane protein (IROMP), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia.

Polypeptides of a parasitic pathogen include but are not limited to the surface antigens of Ichthyophthirius.

A cancer antigen as used herein is a compound, such as a peptide or protein, associated with a tumor or cancer cell surface and which is capable of provoking an immune response when expressed on the surface of an antigen presenting cellin the context of an MHC molecule. Cancer antigens can be prepared from cancer cells either by preparing crude extracts of cancer cells, for example, as described in Cohen, et al., 1994, Cancer Research, 54: 1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

The terms “cancer antigen” and “tumor antigen” are used interchangeably and refer to antigens that are differentially expressed by cancer cells and can thereby be exploited in order to target cancer cells. Cancer antigens are antigens that can potentially stimulate apparently tumor-specific immune responses. Some of these antigens are encoded, although not necessarily expressed, by normal cells. These antigens can be characterized as those that are normally silent (i.e., not expressed) in normal cells, those that are expressed only at certain stages of differentiation, and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as, for example, those carried on RNA and DNA tumor viruses. Examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)—C017—1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

Cancers or tumors and tumor-antigens associated with such tumors (but not exclusively), include acute lymphoblastic leukemia (etv6; aml1; cyclophilin b), B cell lymphoma (Ig-idiotype), glioma (E-cadherin; α-catenin; β-catenin; γ-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)—C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkins lymphoma (Imp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins), testicular cancer (NY-ESO-1), T cell leukemia (HTLV-1 epitopes), and melanoma (Melan-A/MART-1; cdc27; MAGE-3; p21ras; gp100 Pmel117).

Examples of tumor antigens which bind to either or both MHC class I and MHC class II molecules are known in the art. These antigens as well as others are disclosed in PCT Application PCT/US98/18601.

Other cancer antigens that can be used together with the IRM compounds are provided in U.S. Patent Publication No. U.S. 2002/0156033.

Methods for providing prophylactic and/or therapeutic treatments for certain cancers are described, for example, in U.S. Patent Publication Nos. U.S. 2003/0161797; U.S. 2004/0091491; U.S. 2004/0175336; and U.S. 2004/0192585, and U.S. patent application Ser. No. 10/933,594, filed Sep. 3, 2004.

An “allergen” as used herein is a molecule capable of provoking an immune response characterized by production of IgE. An allergen is a substance that can induce an allergic or asthmatic response in a susceptible subject. Thus, in the context of this invention, the term allergen means a specific type of antigen that can trigger an allergic response that is mediated by IgE antibody. The method and preparations of this invention extend to a broad class of such allergens and fragments of allergens or haptens acting as allergens. The list of allergens is enormous and can include pollens, insect venoms, animal dander dust, fungal spores and drugs (e.g. penicillin).

Other allergens that can be used together with the IRM compounds are provided in U.S. Patent Publication No. U.S. 2003/0087848.

The antigen may be an antigen that is encoded by a nucleic acid vector or it may not be encoded in a nucleic acid vector. In the former case the nucleic acid vector is administered to the subject and the antigen is expressed in vivo. In the latter case the antigen may be administered directly to the subject. An antigen not encoded in a nucleic acid vector as used herein refers to any type of antigen that is not a nucleic acid. For instance, in some aspects of the invention the antigen not encoded in a nucleic acid vector is a peptide or a polypeptide. Minor modifications of the primary amino acid sequences of peptide or polypeptide antigens may also result in a polypeptide that has substantially equivalent antigenic activity as compared to the unmodified counterpart polypeptide. Such modifications may be deliberate, as by site-directed mutagenesis, or may be spontaneous. All of the polypeptides produced by these modifications are included herein as long as antigenicity still exists. The peptide or polypeptide may be, for example, virally derived. The antigens useful in the invention may be any length, ranging from small peptide fragments of a full length protein or polypeptide to the full length form. For example, the antigen may be less than 5, less than 8, less than 10, less than 15, less than 20, less than 30, less than 50, less than 70, less than 100, or more amino acid residues in length, provided it stimulates a specific immune response when used in combination with the IRM compounds and/or other agents of the invention.

The nucleic acid encoding the antigen is operatively linked to a gene expression sequence that directs the expression of the antigen nucleic acid within a eukaryotic cell. The gene expression sequence is any regulatory nucleotide sequence, such as a promoter sequence or promoter-enhancer combination, which facilitates the efficient transcription and translation of the antigen nucleic acid to which it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, b-actin promoter and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined antigen nucleic acid. The gene expression sequences optionally include enhancer sequences or upstream activator sequences as desired.

The antigen nucleic acid is operatively linked to the gene expression sequence. As used herein, the antigen nucleic acid sequence and the gene expression sequence are said to be operably linked when they are covalently linked in such away as to place the expression or transcription and/or translation of the antigen coding sequence under the influence or control of the gene expression sequence. Two DNA sequences are said to be operably linked if induction of a promoter in the 5′ gene expression sequence results in the transcription of the antigen sequence and if the nature of the linkage between the two DNA sequences does not (1) result in the introduction of a frame-shift mutation, (2) interfere with the ability of the promoter region to direct the transcription of the antigen sequence, or (3) interfere with the ability of the corresponding RNA transcript to be translated into a protein. Thus, a gene expression sequence would be operably linked to an antigen nucleic acid sequence if the gene expression sequence were capable of effecting transcription of that antigen nucleic acid sequence such that the resulting transcript is translated into the desired protein or polypeptide.

The antigen nucleic acid of the invention may be delivered to the immune system alone or in association with a vector. In its broadest sense, a vector is any vehicle capable of facilitating the transfer of the antigen nucleic acid to the cells of the immune system so that the antigen can be expressed and presented on the surface of the immune cell. The vector generally transports the nucleic acid to the immune cells with reduced degradation relative to the extent of degradation that would result in the absence of the vector. The vector optionally includes the above-described gene expression sequence to enhance expression of the antigen nucleic acid in immune cells. In general, the vectors useful in the invention include, but are not limited to, plasmids, phagemids, viruses, and other vehicles derived from viral or bacterial sources that have been manipulated by the insertion or incorporation of the antigen nucleic acid sequences. Viral vectors are a preferred type of vector and include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known in the art.

Preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., Gene Transfer and Expression, A Laboratory Manual W. H. Freeman C. O., New York (1990) and Murray, E. J. Methods in Molecular Biology, vol. 7, Humana Press, Inc., Cliffton, N.J. (1991).

A preferred virus for certain applications is the adeno-associated virus, a double-stranded DNA virus. The adeno-associated virus can be engineered to be replication-deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, wild-type adeno-associated virus manifest some preference for integration sites into human cellular DNA, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Recombinant adeno-associated viruses that lack the replicase protein apparently lack this integration sequence specificity.

Other vectors include plasmid vectors. Plasmid vectors have been extensively described in the art and are well known to those of skill in the art. See e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRc/CMV, SV40, and pBlueScript. Other plasmids are well known to those of ordinary skill in the art. Additionally, plasmids may be custom designed using restriction enzymes and ligation reactions to remove and add specific fragments of DNA.

A gene-carrying plasmid can be delivered to the immune system using bacteria. Modified forms of bacteria such as Salmonella can be transfected with the plasmid and used as delivery vehicles. The bacterial delivery vehicles can be administered to a host subject orally or by other administration means. The bacteria deliver the plasmid to immune cells, e.g. B cells, dendritic cells, likely by passing through the gut barrier. High levels of immune protection have been established using this methodology. Such methods of delivery are useful for the aspects of the invention utilizing systemic delivery of antigen, IRM compounds and/or other therapeutic agent.

In some aspects of the invention, the IRM compounds are administered along with therapeutic agents such as disorder-specific medicaments. As used herein, a disorder-specific medicament is a therapy or agent that is used predominately in the treatment or prevention of a disorder. In one aspect, the IRM compounds may be administered to a subject with an anti-microbial agent. An anti-microbial agent, as used herein, refers to a naturally occurring or synthetic compound that is capable of killing or inhibiting infectious organisms. The type of anti-microbial agent useful according to the invention will depend upon the type of organism with which the subject is infected or at risk of becoming infected.

In one aspect, the invention provides a method for treating or preventing a disorder. The method involves the administration of a synergistic combination of an IRM compound and a disorder-specific medicament in an effective amount to prevent or treat the disorder to a subject having in need of such treatment.

In one aspect, the combination of IRM compounds and disorder-specific such treatment medicaments allows for the administration of higher doses of disorder-specific medicaments without as, many side effects as are ordinarily experienced at those high doses. In another aspect, the combination of IRM compounds and disorder-specific medicaments allows for the administration of lower, sub-therapeutic doses of either compound, but with higher efficacy than would otherwise be achieved using such low doses. As one example, by administering a combination of an IRM compound and a medicament, it is possible to achieve an effective response even though the medicament is administered at a dose that, if administered alone, would not provide a therapeutic benefit (i.e., a sub-therapeutic dose). As another example, the combined administration achieves a response even though the IRM compound is administered at a dose that, if given alone, would not provide a therapeutic benefit.

The IRM compounds can also be administered on fixed schedules or in different temporal relationships to one another. The various combinations have many advantages over the prior art methods of modulating immune responses or preventing or treating disorders, particularly with regard to decreased non-specific toxicity to normal tissues.

The invention encompasses the administration of the IRM compounds along with a disorder-specific medicament in order to provide a synergistic effect useful in the prevention and/or treatment of a disorder. The beneficial effects of the IRM compounds are due, in part, to the modulation and stimulation of T_{H}1 immune responses by these agents. The imidazoquinolines of the invention may provide the synergistic response via a number of mechanisms, including but not so limited to stimulation of hemopoietic recovery during or following cancer therapy, anti-microbial infection activity, enhancement of uptake of disorder-specific medicaments by immune cells and non-immune cells (depending upon the nature of the medicament), and inhibition or prevention of allergic responses to allergens in general and more specifically to the medicament.

Beyond the synergistic effect of co-administering an IRM compound and a disorder-specific medicament, another order of synergistic immune response can be obtained by administering, in combination, an IRM compound, a disorder-specific medicament, and an agonist of a member of either the TNF Superfamily or TNFR Superfamily, as described in, for example, U.S. Patent Publication No. U.S. 2004/0141950.

The IRM compounds function to enhance defense mechanisms against bacterial, fungal, parasitic and viral infections. The prevention and control of such infections in immunocompromised cancer patients is a major challenge in the treatment and management of the disease. Such infections can usually disadvantageously delay or alter the course of treatment for cancer patients. The cellular and humoral immune responses stimulated by the nucleic acids reflect the body's own natural defense system against invading pathogens. The IRM compounds perform this function through the activation of innate immunity, which is known to be most effective in the elimination of microbial infections. Enhancement of innate immunity occurs, inter alia, via increased IFN-α production and increased NK cell activity, both of which are effective in the treatment of microbial infections. The IRM compounds also function by enhancement of antibody-dependent cell cytotoxicity. This latter mechanism provides long-lasting effects of the nucleic acids, thereby reducing dosing regimes, improving compliance and maintenance therapy, reducing emergency situations; and improving quality of life. Some examples of common opportunistic infections in cancer patients are caused by Listeria monocytogenes, Pneumocystis carinii, cytomegalovirus, Mycobacterium tuberculosis, Staphylococcus aureus, Streptococcus pneumoniae, Haemophilus influenzae, Escherichia coli, Klebsiella pneumoniae, Pseudomonas aeruginosa, Nocardia, Candida, Aspergillus, and herpes viruses such as herpes simplex virus.

It is sometimes the case that subjects undergoing cancer treatment experience an adverse allergic reaction to the cancer medicament formulation being administered. The reaction may be specific to the cancer medicament itself or to other substances included in the cancer medicament formulation (e.g., the carrier substance, stabilizing agents, or sterilizing agents within the formulation). An example of a medicament that often triggers an allergic reaction upon administration is a formulation of Taxol. Such a reaction makes the use of such a medicament less desirable, and at the very least, may lead to the administration of the medicament at lower than therapeutic doses in order to avoid the allergic reaction. The present invention provides a method for avoiding such an adverse reaction through the administration of an IRM compound. Reducing or eliminating the allergic reaction altogether may also allow for administration of disorder-specific medicaments in doses greater than the therapeutic dose, or at least greater than the doses currently administered.

The IRM compounds of the invention are also useful in the regulation of adverse allergic reactions in subjects undergoing transfusions. Subjects undergoing cancer treatment often require transfusions of red cells and/or platelets. Either due to incomplete separation of these cell types from others or due to differences in minor histocompatibility loci between the donor and the recipient of these blood products, subjects being infused may experience an acute allergic reaction to the transfusion. To counter this reaction, which is primarily a T_{H}2 type response, patients are administered allergy medication such as antihistamines. Since IRM compounds elicit a T_{H}1 response, the subject may be administered an IRM compound prior to or at the time of the transfusion in order to prevent or diminish the T_{H}2 allergic reaction that might otherwise occur.

The IRM compounds when combined with the asthma/allergy medicaments have many advantages over each composition alone for the treatment of asthma and allergy. The IRM compound functions in some aspects by simultaneously suppressing T_{H}2-type immune responses (IL-4, IgE production, histamine release) that can result in airway inflammation and bronchial spasm, and/or inducing T_{H}1-type immune responses (IFN-γ and IL-12 production) that promote harmless antibody and cellular responses. This creates an environment inside the body that safely and effectively prevents hypersensitive reactions from occurring, thereby eliminating symptoms.

The IRM compounds when used in the methods of the invention can eliminate/reduce bronchial hyper-reactivity, bronchoconstriction, bronchial obstruction, airway inflammation and atopy (which improves asthma control, normalizes lung function, prevents irreversible airway injury); and may also inhibit acute response to exercise, cold dry air, and SO₂. The IRM compounds provide long-lasting effects, thus reducing dosing regimes, improving compliance and maintenance therapy, reducing emergency situations; and improving quality of life. These compounds are also useful because they provide early anti-infective activity, which leads to decreasing infectious episodes, which further reduces hyper-reactive immune responses. This is especially true in subjects like children or immuno-compromised subjects. Furthermore, use of the IRM compounds reduces/eliminates use of inhalers, which can exacerbate hypersensitive reactions, by providing simpler and safer delivery and by allowing less drug to be used.

Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”,“anti-fungal agent”, “anti-parasitic agent” and “parasiticide” have well-established meanings to those of ordinary skill in the art and are defined in standard medical texts. Anti-bacterial agents kill or inhibit bacteria, and include antibiotics as well as other synthetic or natural compounds having similar functions. Antibiotics are low molecular weight molecules that are produced as secondary metabolites by cells, such as microorganisms. In general, antibiotics interfere with one or more bacterial functions or structures that are specific for the microorganism and which are not present in host cells. Anti-viral agents, which can be isolated from natural sources or synthesized, are useful for killing or inhibiting viruses. Anti-fungal agents are used to treat superficial fungal infections as well as opportunistic and primary systemic fungal infections. Anti-parasite agents kill or inhibit parasites.

One of the problems with anti-infective therapies is the side effects occurring in the host that is treated with the anti-infective. For instance, many anti-infectious agents can kill or inhibit a broad spectrum of microorganisms and are not specific for a particular type of species. Treatment with these types of anti-infectious agents results in the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host.

Another problem with widespread use of anti-infectants is the development of antibiotic resistant strains of microorganisms. Already, vancomycin-resistant enterococci, penicillin-resistant pneumococci, multi-resistant S. aureus, and multi-resistant tuberculosis strains have developed and are becoming major clinical problems. Widespread use of anti-infectants will likely produce many antibiotic-resistant strains of bacteria. As a result, new anti-infective strategies will be required to combat these microorganisms.

A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class Gram-positive or Gram-negative. These types of antibiotics are referred to as narrow spectrum antibiotics. Other antibiotics, which are effective against a single organism or disease and not against other types of bacteria, are referred to as limited spectrum antibiotics.

Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors. Cell wall synthesis inhibitors inhibit a step in the process of cell wall synthesis, and in general in the synthesis of bacterial peptidoglycan. Cell wall synthesis inhibitors include β-lactam antibiotics, natural penicillins, semi-synthetic penicillins, ampicillin, clavulanic acid, cephalolsporins, and bacitracin.

The β-lactams are antibiotics containing a four-membered β-lactam ring, which inhibits the last step of peptidoglycan synthesis. The β-lactam antibiotics produced by penicillium are the natural penicillins, such as penicillin G or penicillin V. The natural penicillins have a narrow spectrum of activity and are generally effective against Streptococcus, Gonococcus, and Staphylococcus. Other types of natural penicillins, which are also effective against Gram-positive bacteria, include penicillins F, X, K, and O.

Semi-synthetic penicillins are generally modifications of the molecule 6-aminopenicillanic acid produced by a mold. The 6-aminopenicillanic acid can be modified by addition of side chains that produce penicillins having broader spectrums of activity than natural penicillins or various other advantageous properties. Some types of semi-synthetic penicillins have broad spectrums against Gram-positive and Gram-negative bacteria, but are inactivated by penicillinase. These semi-synthetic penicillins include ampicillin, carbenicillin, oxacillin, azlocillin, mezlocillin, and piperacillin. Other types of semi-synthetic penicillins have narrower activities against Gram-positive bacteria, but have developed properties such that they are not inactivated by penicillinase. These include, for instance, methicillin, dicloxacillin, and nafcillin. Some of the broad spectrum semi-synthetic penicillins can be used in combination with β-lactamase inhibitors, such as clavulamic acids and sulbactam. The β-lactamase inhibitors do not have anti-microbial action but they function to inhibit penicillinase, thus protecting the semi-synthetic penicillin from degradation.

One of the serious side effects associated with penicillins, both natural and semi-synthetic, is penicillin-allergy. Penicillin allergies are very serious and can cause death rapidly. In a subject that is allergic to penicillin, the β-lactam molecule will attach to a serum protein that initiates an IgE-mediated inflammatory response. The inflammatory response leads to anaphylaxis and possibly death.

Another type of β-lactam antibiotic is the cephalolsporins. They are sensitive to degradation by bacterial β-lactamases, and thus, are not always effective alone. Cephalolsporins, however, are resistant to penicillinase. They are effective against a variety of Gram-positive and Gram-negative bacteria. Cephalolsporins include, but are not limited to, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, and moxalactam.

Bacitracin is another class of antibiotics that inhibit cell wall synthesis. Although bacitracin is effective against Gram-positive bacteria, its use is limited in general to topical administration because of its high toxicity. Since lower effective doses of bacitracen can be used when the compound is administered with the IRM compounds of the invention, this compound can be used systemically and the toxicity reduced.

Carbapenems are another broad spectrum β-lactam antibiotic, which is capable of inhibiting cell wall synthesis. Examples of carbapenems include, but are not limited to, imipenems. Monobactems are also broad spectrum β-lactam antibiotics, and include, euztreonam. An antibiotic produced by streptomyces, vancomycin, is also effective against Gram-positive bacteria by inhibiting cell membrane synthesis.

Another class of anti-bacterial agents is the anti-bacterial agents that are cell membrane inhibitors. These compounds disorganize the structure or inhibit the function of bacterial membranes. One problem with anti-bacterial agents that are cell membrane inhibitors is that they can produce effects in eukaryotic cells as well as bacteria because of the similarities in phospholipids in bacterial and eukaryotic membranes. Thus these compounds are rarely specific enough to permit these compounds to be used systemically and prevent the use of high doses for local administration.

One clinically cell membrane inhibitor is Polymyxin. Polymyxin is effective mainly against Gram-negative bacteria and is generally used in severe Pseudomonas infections or Pseudomonas infections that are resistant to less toxic antibiotics. The severe side effects associated with systemic administration of this compound include damage to the kidney and other organs.

Other cell membrane inhibitors include Amphotericin B and Nystatin, which are also anti-fungal agents used predominantly in the treatment of systemic fungal infections and Candida yeast infections, respectively. Imidazoles are another class of antibiotic that is a cell membrane inhibitor. Imidazoles are used as bacterial agents as well as anti-fungal agents, e.g., used for treatment of yeast infections, dermatophytic infections, and systemic fungal infections. Imidazoles include but are not limited to clotrimazole, miconazole, ketoconazole, itraconazole, and fluconazole.

Many anti-bacterial agents are protein synthesis inhibitors. These compounds prevent bacteria from synthesizing structural proteins and enzymes and thus cause inhibition of bacterial cell growth or function or cell death. Anti-bacterial agents that block transcription include but are not limited to Rifampins and Ethambutol. Rifampins, which inhibit the enzyme RNA polymerase, have a broad spectrum activity and are effective against Gram-positive and Gram-negative bacteria as well as Mycobacterium tuberculosis. Ethambutol is effective against Mycobacterium tuberculosis.

Anti-bacterial agents that block translation include but are not limited to tetracyclines, chloramphenicol, the macrolides (e.g., erythromycin) and the aminoglycosides (e.g., streptomycin).

The aminoglycosides are a class of antibiotics that are produced by the bacterium Streptomyces, such as, for instance streptomycin, kanamycin, tobramycin, amikacin, and gentamicin. Aminoglycosides have been used against a wide variety of bacterial infections caused by Gram-positive and Gram-negative bacteria. Streptomycin has been used extensively as a primary drug in the treatment of tuberculosis. Gentamicin is used against many strains of Gram-positive and Gram-negative bacteria, including Pseudomonas infections, especially in combination with Tobramycin. Kanamycin is used against many Gram-positive bacteria, including penicillin-resistant Staphylococci. One side effect of aminoglycosides that has limited their use clinically is that at dosages that are essential for efficacy, prolonged use has been shown to impair kidney function and cause damage to the auditory nerves leading to deafness.

Another type of translation inhibitor anti-bacterial agent is the tetracyclines. The tetracyclines are a class of antibiotics that are broad-spectrum and are effective against a variety of Gram-positive and Gram-negative bacteria. Examples of tetracyclines include tetracycline, minocycline, doxycycline, and chlortetracycline. They are important for the treatment of many types of bacteria but are particularly important in the treatment of Lyme disease. As a result of their low toxicity and minimal direct side effects, the tetracyclines have been overused and misused by the medical community, leading to problems. For instance, their overuse has led to widespread development of resistance. When used in combination with the IRM compounds of the invention, these problems can be minimized and tetracyclines can be effectively used for the broad-spectrum treatment of many bacteria.

Anti-bacterial agents such as the macrolides bind reversibly to the 50S ribosomal subunit and inhibit elongation of the protein by peptidyl transferase or prevent the release of uncharged tRNA from the bacterial ribosome or both. These compounds include erythromycin, roxithromycin, clarithromycin, oleandomycin, and azithromycin. Erythromycin is active against most Gram-positive bacteria, Neisseria, Legionella and Haemophilus, but not against the Enterobacteriaceae. Lincomycin and clindamycin, which block peptide bond formation during protein synthesis, are used against Gram-positive bacteria.

Another type of translation inhibitor is chloramphenicol. Chloramphenicol binds the 70S ribosome inhibiting the bacterial enzyme peptidyl transferase thereby preventing the growth of the polypeptide chain during protein synthesis. One serious side effect associated with chloramphenicol is aplastic anemia. Aplastic anemia develops at doses of chloramphenicol, which are effective for treating bacteria in a small proportion (1/50,000) of patients. Chloramphenicol, which was once a highly prescribed antibiotic, is now seldom uses as a result of the deaths from anemia. Because of its effectiveness it is still used in life-threatening situations (e.g. typhoid fever). By combining chloramphenicol with the IRM compounds these compounds can again be used as anti-bacterial agents because the immunostimulatory agents allow a lower dose of the chloramphenicol to be used, a dose that does not produce side effects.

Some anti-bacterial agents disrupt nucleic acid synthesis or function, e.g., bind to DNA or RNA so that their messages cannot be read. These include but are not limited to quinolones and co-trimoxazole, both synthetic chemicals and rifamycins, a natural or semi-synthetic chemical. The quinolones block bacterial DNA replication by inhibiting the DNA gyrase, the enzyme needed by bacteria to produce their circular DNA. They are broad spectrum and examples include norfloxacin, ciprofloxacin, enoxacin, nalidixic acid and temafloxacin. Nalidixic acid is a bactericidal agent that binds to the DNA gyrase enzyme (topoisomerase), which is essential for DNA replication and allows supercoils to be relaxed and reformed, inhibiting DNA gyrase activity. The main use of nalidixic acid is in treatment of lower urinary tract infections (UTI) because it is effective against several types of Gram-negative bacteria such as E. coli, Enterobacter aerogenes, K. pneumoniae and Proteus species, which are common causes of UTI. Co-trimoxazole is a combination of sulfamethoxazole and trimethoprim, which blocks the bacterial synthesis of folic acid needed to make DNA nucleotides. Rifampicin is a derivative of rifamycin that is active against Gram-positive bacteria (including Mycobacterium tuberculosis and meningitis caused by Neisseria meningitidis) and some Gram-negative bacteria. Rifampicin binds to the P subunit of the polymerase and blocks the addition of the first nucleotide, which is necessary to activate the polymerase, and thereby blocks mRNA synthesis.

Another class of anti-bacterial agents is compounds that function as competitive inhibitors of bacterial enzymes. The competitive inhibitors are mostly all structurally similar to a bacterial growth factor and compete for binding but do not perform the metabolic function in the cell. These compounds include sulfonamides and chemically modified forms of sulfanilamide, which have even higher and broader antibacterial activity. The sulfonamides (e.g. gantrisin and trimethoprim) are useful for the treatment of Streptococcus pneumoniae, β-hemolytic streptococci and E. coli, and have been used in the treatment of uncomplicated UTI caused by E. coli, and in the treatment of meningococcal meningitis.

Anti-viral agents are compounds that prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection that can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Another category of anti-viral agents are nucleotide analogues. Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine).

Another class of anti-viral agents are cytokines such as interferons. The interferons are cytokines which are secreted by virus-infected cells as well as immune cells. The interferons function by binding to specific receptors on cells adjacent to the infected cells, causing the change in the cell that protects it from infection by the virus. Type I interferons (e.g., IFN-α and/or IFN-β) also induce the expression of Class I and Class II MHC molecules on the surface of infected cells, resulting in increased antigen presentation for host immune cell recognition. Type I interferons are available as recombinant forms and have been used for the treatment of chronic hepatitis B and C infection. At the dosages that are effective for anti-viral therapy, interferons have severe side effects such as fever, malaise and weight loss.

Immunoglobulin therapy is used for the prevention of viral infection. Immunoglobulin therapy for viral infections is different than bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general there are two types of immunoglobulin therapies, normal immunoglobulin therapy and hyper-immunoglobulin therapy. Normal immune globulin therapy utilizes an antibody product that is prepared from the serum of normal blood donors and pooled. This pooled product contains low titers of antibody to a wide range of human viruses, such as hepatitis A, parvovirus, or enterovirus (especially in neonates). Hyper-immune globulin therapy utilizes antibodies that are prepared from the serum of individuals who have high titers of an antibody to a particular virus. Those antibodies are then used against a specific virus. Examples of hyper-immune globulins include zoster immune globulin (useful for the prevention of varicella in immuno-compromised children and neonates), human rabies immunoglobulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

Another type of immunoglobulin therapy is active immunization. This involves the administration of antibodies or antibody fragments to viral surface proteins. Two types of vaccines that are available for active immunization of hepatitis B include serum-derived hepatitis B antibodies and recombinant hepatitis B antibodies. Both are prepared from HBsAg. The antibodies are administered in three doses to subjects at high risk of infection with hepatitis B virus, such as health care workers, sexual partners of chronic carriers, and infants.

The combination of IRM compounds with immunoglobulin therapy also provides benefit via the ability of certain IRM compounds to enhance antibody-dependent cellular cytotoxicity (ADCC).

Anti-fungal agents are useful for the treatment and prevention of infective fungi. Anti-fungal agents are sometimes classified by their mechanism of action. Some anti-fungal agents function as cell wall inhibitors by inhibiting glucose synthase. Other anti-fungal agents function by destabilizing membrane integrity.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, imidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

The IRM compounds may also be administered in conjunction with an anti-cancer therapy. Anti-cancer therapies include cancer medicaments, radiation and surgical procedures. As used herein, a “cancer medicament” refers to an agent that is administered to a subject for the purpose of treating a cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

Cancer is currently treated using a variety of modalities including surgery, radiation therapy and chemotherapy. The choice of treatment modality will depend upon the type, location and dissemination of the cancer. For example, surgery and radiation therapy may be more appropriate in the case of solid well-defined tumor masses and less practical in the case of non-solid tumor cancers such as leukemia and lymphoma. One of the advantages of surgery and radiation therapy is the ability to control to some extent the impact of the therapy, and thus to limit the toxicity to normal tissues in the body. However, surgery and radiation therapy are often followed by chemotherapy to guard against any remaining or radio-resistant cancer cells. Chemotherapy is also the most appropriate treatment for disseminated cancers such as leukemia and lymphoma as well as metastases.

Chemotherapy refers to therapy using chemical and/or biological agents to attack cancer cells. Unlike localized surgery or radiation, chemotherapy is generally administered in a systemic fashion and thus toxicity to normal tissues is a major concern. Because many chemotherapy agents target cancer cells based on their proliferative profiles, tissues such as the gastrointestinal tract and the bone marrow, which are normally proliferative, are also susceptible to the effects of the chemotherapy. One of the major side effects of chemotherapy is myelosuppression (including anemia, neutropenia and thrombocytopenia), which results from the death of normal hemopoietic precursors.

Many chemotherapeutic agents have been developed for the treatment of cancer. Not all tumors, however, respond to chemotherapeutic agents and others although initially responsive to chemotherapeutic agents may develop resistance. As a result, the search for effective anti-cancer drugs has intensified in an effort to find even more effective agents with less non-specific toxicity.

Cancer medicaments function in a variety of ways. Some cancer medicaments work by targeting physiological mechanisms that are specific to tumor cells. Examples include the targeting of specific genes and their gene products (e.g., proteins), which are mutated in cancers. Such genes include but are not limited to oncogenes (e.g., Ras, Her2, bcl-2), tumor suppressor genes (e.g., EGF, p53, Rb), and cell cycle targets (e.g., CDK4, p21, telomerase). Cancer medicaments can alternately target signal transduction pathways and molecular mechanisms that are altered in cancer cells. Targeting of cancer cells via the epitopes expressed on their cell surface is accomplished through the use of monoclonal antibodies. This latter type of cancer medicament is generally referred to herein as immunotherapy.

Other cancer medicaments target cells other than cancer cells. For example, some medicaments prime the immune system to attack tumor cells (i.e., cancer vaccines). Still other medicaments, called angiogenesis inhibitors, function by attacking the blood supply of solid tumors. Since the most malignant cancers are able to metastasize (i.e., exist the primary tumor site and seed a distal tissue, thereby forming a secondary tumor), medicaments that impede this metastasis are also useful in the treatment of cancer. Angiogenic mediators include basic FGF, VEGF, angiopoietins, angiostatin, endostatin, TNF-α, TNP-470, thrombospondin-1, platelet factor 4, CAI, and certain members of the integrin family of proteins. One category of this type of medicament is a metalloproteinase inhibitor, which inhibits the enzymes used by the cancer cells to exist the primary tumor site and extravasate into another tissue.

Some cancer cells are antigenic and thus can be targeted by the immune system. In one aspect, the combined administration of IRM compounds and cancer medicaments, particularly those that are classified as cancer immunotherapies, is useful for stimulating a specific immune response against a cancer antigen.

The theory of immune surveillance is that a prime function of the immune system is to detect and eliminate neoplastic cells before a tumor forms. A basic principle of this theory is that cancer cells are antigenically different from normal cells and thus elicit immune reactions that are similar to those that cause rejection of immunologically incompatible allografts. Studies have confirmed that tumor cells differ, either qualitatively or quantitatively, in their expression of antigens. For example, “tumor-specific antigens” are antigens that are specifically associated with tumor cells but not normal cells. Examples of tumor specific antigens are viral antigens in tumors induced by DNA or RNA viruses. “Tumor-associated” antigens are present in both tumor cells and normal cells but are present in a different quantity or a different form in tumor cells. Examples of such antigens are oncofetal antigens (e.g., carcinoembryonic antigen), differentiation antigens (e.g., T and Tn antigens), and oncogene products (e.g., HER/neu).

Different types of cells that can kill tumor targets in vitro and in vivo have been identified: natural killer cells (NK cells), cytolytic T lymphocytes (CTLs), lymphokine-activated killer cells (LAKs), and activated macrophages. NK cells can kill tumor cells without having been previously sensitized to specific antigens, and the activity does not require the presence of class I antigens encoded by the major histocompatibility complex (MHC) on target cells. NK cells are thought to participate in the control of nascent tumors and in the control of metastatic growth. In contrast to NK cells, CTLs can kill tumor cells only after they have been sensitized to tumor antigens and when the target antigen is expressed on the tumor cells that also express MHC class I. CTLs are thought to be effector cells in the rejection of transplanted tumors and of tumors caused by DNA viruses. LAK cells are a subset of null lymphocytes distinct from the NK and CTL populations. Activated macrophages can kill tumor cells in a manner that is not antigen dependent nor MHC restricted once activated. Activated macrophages are through to decrease the growth rate of the tumors they infiltrate. In vitro assays have identified other immune mechanisms such as antibody-dependent, cell-mediated cytotoxic reactions and lysis by antibody plus complement. However, these immune effector mechanisms are thought to be less important in vivo than the function of NK, CTLs, LAK, and macrophages in vivo (for review see Piessens, W. F., and David, J., “Tumor Immunology”, In: Scientific American Medicine, Vol. 2, Scientific American Books, N.Y., pp. 1-13, 1996.

The goal of immunotherapy is to augment a patient's immune response to an established tumor. One method of immunotherapy includes the use of adjuvants. Adjuvant substances derived from microorganisms, such as bacillus Calmette-Guerin, heighten the immune response and enhance resistance to tumors in animals.

Immunotherapeutic agents are medicaments that derive from antibodies or antibody fragments that specifically bind or recognize a cancer antigen. Antibody-based immunotherapies may function by binding to the cell surface of a cancer cell and thereby stimulate the endogenous immune system to attack the cancer cell. Antibody-based therapy also can function as a delivery system for the specific targeting of toxic substances to cancer cells. Antibodies are usually conjugated to toxins such as ricin (e.g., from castor beans), calicheamicin and maytansinoids, to radioactive isotopes such as Iodine-131 and Yttrium-90, to chemotherapeutic agents (as described herein), or to biological response modifiers. In this way, the toxic substances can be concentrated in the region of the cancer and non-specific toxicity to normal cells can be minimized. In addition to the use of antibodies that are specific for cancer antigens, antibodies that bind to vasculature, such as those that bind to endothelial cells, are also useful in the invention. This is because generally solid tumors are dependent upon newly formed blood vessels to survive, and thus most tumors are capable of recruiting and stimulating the growth of new blood vessels. As a result, one strategy of many cancer medicaments is to attack the blood vessels feeding a tumor and/or the connective tissues (or stroma) supporting such blood vessels.

The use of IRM compounds in conjunction with immunotherapeutic agents such as monoclonal antibodies is able to increase long-term survival through a number of mechanisms including significant enhancement of ADCC, activation of natural killer (NK) cells and an increase in IFN-α levels. The IRM compounds when used in combination with monoclonal antibodies serve to reduce the dose of the antibody required to achieve a biological result.

Cancer vaccines are medicaments that are intended to stimulate an endogenous immune response against cancer cells. Currently produced vaccines predominantly activate the humoral immune system (i.e., the antibody-dependent immune response). Other vaccines currently in development are focused on activating the cell-mediated immune system including cytotoxic T lymphocytes that are capable of killing tumor cells. Cancer vaccines generally enhance the presentation of cancer antigens to both antigen presenting cells (e.g., macrophages and dendritic cells) and/or to other immune cells such as T cells, B cells, and NK cells.

Although cancer vaccines may take one of several forms, as discussed infra, their purpose is to deliver cancer antigens and/or cancer associated antigens to antigen presenting cells (APC) in order to facilitate the endogenous processing of such antigens by APC and the ultimate presentation of antigen presentation on the cell surface in the context of MHC class I molecules. One form of cancer vaccine is a whole cell vaccine that is a preparation of cancer cells that have been removed from a subject, treated ex vivo and then reintroduced as whole cells in the subject. Lysates of tumor cells can also be used as cancer vaccines to elicit an immune response. Another form cancer vaccine is a peptide vaccine that uses cancer-specific or cancer-associated small proteins to activate T cells. Cancer-associated proteins are proteins that are not exclusively expressed by cancer cells (i.e., other normal cells may still express these antigens). However, the expression of cancer-associated antigens is generally consistently upregulated with cancers of a particular type. Yet another form of cancer vaccine is a dendritic cell vaccine that includes whole dendritic cells that have been exposed to a cancer antigen or a cancer-associated antigen in vitro. Lysates or membrane fractions of dendritic cells may also be used as cancer vaccines. Dendritic cell vaccines are able to activate antigen-presenting cells directly. Other cancer vaccines include ganglioside vaccines, heat-shock protein vaccines, viral and bacterial vaccines, and nucleic acid vaccines.

The use of IRM compounds in conjunction with cancer vaccines provides an improved antigen-specific humoral and cell mediated immune response, in addition to activating NK cells and endogenous dendritic cells, and increasing IFN-α levels. This enhancement allows a vaccine with a reduced antigen dose to be used to achieve the same beneficial effect. In some instances, cancer vaccines may be used along with adjuvants, such as those described above.

Other vaccines take the form of dendritic cells (DCs) that have been exposed to cancer antigens in vitro, have processed the antigens and are able to express the cancer antigens at their cell surface in the context of MHC molecules for effective antigen presentation to other immune system cells. In one embodiment, the IRM compound and the DC vaccine are mixed upon re-injection into a subject. Alternatively, the IRM compound can be used in the in vitro preparation of the vaccine for example in the culture, maturation or activation of DCs. Monocytic DCs (mDCs) in particular can benefit from the combined use of IRM compounds. Synergy when using mixed populations of CDs (i.e., combinations of plasmacytoid DCs (pDCs) and mDCs) is also envisioned.

The IRM compounds are used in one aspect of the invention in conjunction with cancer vaccines that are dendritic cell based. A dendritic cell is a professional antigen presenting cell. Dendritic cells form the link between the innate and the acquired immune system by presenting antigens and through their expression of pattern recognition receptors that detect microbial molecules like LPS in their local environment. Dendritic cells efficiently internalize, process, and present soluble specific antigen to which it is exposed. The process of internalizing and presenting antigen causes rapid upregulation of the expression of major histocompatibility complex (MHC) and costimulatory molecules, the production of cytokines, and migration toward lymphatic organs where they are believed to be involved in the activation of T cells.

As used herein, chemotherapeutic agents embrace all other forms of cancer medicaments that do not fall into the categories of immunotherapeutic agents or cancer vaccines. Chemotherapeutic agents as used herein encompass both chemical and biological agents. These agents function to inhibit a cellular activity that the cancer cell is dependent upon for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. Combination chemotherapy and IRM compound administration increases the maximum tolerable dose of chemotherapy.

Further examples of cancer medicaments that can be used in the methods and compositions of the present invention are listed in U.S. Patent Publication No. U.S. 2002/0156033.

The IRM compounds may also be administered in conjunction with an asthma or allergy medicament. An “asthma/allergy medicament” as used herein is a composition of matter that reduces the symptoms, inhibits the asthmatic or allergic reaction, or prevents the development of an allergic or asthmatic reaction. Various types of medicaments for the treatment of asthma and allergy are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below. In most embodiments the asthma/allergy medicament is useful to some degree for treating both asthma and allergy.

Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and medacromil; long-acting bronchodilators, such as long-acting β₂-agonists and methylxanthines; and leukotriene modifiers. The quick-relief medications include short-acting β₂-agonists, anti-cholinergics, and systemic corticosteroids. There are many side effects associated with each of these drugs and none of the drugs alone or in combination is capable of preventing or completely treating asthma.

Asthma medicaments include, but are not limited, PDE-4 inhibitors, Bronchodilator/β₂-agonists, K⁺ channel openers, VLA-4 antagonists, Neurokin antagonists, TXA2 synthesis inhibitors, Xanthanines, Arachidonic acid antagonists, 5-lipoxygenase inhibitors, Thromboxin A2 receptor antagonists, Thromboxane A2 antagonists, Inhibitor of 5-lipox activation proteins, and Protease inhibitors.

Bronchodilator/β₂-agonists are a class of compounds that cause bronchodilation or smooth muscle relaxation. Bronchodilator/β₂-agonists include, but are not limited to, salmeterol, salbutamol, albuterol, terbutaline, D2522/formoterol, fenoterol, bitolterol, pirbuerol methylxanthines and orciprenaline. Long-acting β₂-agonists and bronchodilators are compounds that are used for long-term prevention of symptoms in addition to the anti-inflammatory therapies. Long-acting β₂-agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. Dose-related acute toxicities are a particular problem with these types of compounds. As a result, routine serum concentration must be monitored in order to account for the toxicity and narrow therapeutic range arising from individual differences in metabolic clearance. Side effects include tachycardia, nausea and vomiting, tachyarrhythmias, central nervous system stimulation, headache, seizures, hematemesis, hyperglycemia and hypokalemia. Short-acting β₂-agonists include, but are not limited to, albuterol, bitolterol, pirbuterol, and terbutaline. Some of the adverse effects associated with the administration of short-acting β₂-agonists include tachycardia, skeletal muscle tremor, hypokalemia, increased lactic acid, headache, and hyperglycemia.

Conventional methods for treating or preventing allergy have involved the use of antihistamines or desensitization therapies. Antihistamines and other drugs which block the effects of chemical mediators of the allergic reaction help to regulate the severity of the allergic symptoms but do not prevent the allergic reaction and have no effect on subsequent allergic responses. Desensitization therapies are performed by giving small doses of an allergen, usually by injection under the skin, in order to induce an IgG-type response against the allergen. The presence of IgG antibody helps to neutralize the production of mediators resulting from the induction of IgE antibodies, it is believed. Initially, the subject is treated with a very low dose of the allergen to avoid inducing a severe reaction and the dose is slowly increased. This type of therapy is dangerous because the subject is actually administered the compounds which cause the allergic response and severe allergic reactions can result.

Allergy medicaments include, but are not limited to, antihistamines, steroids, and prostaglandin inducers. Antihistamines are compounds that counteract histamine released by mast cells or basophils. These compounds are well known in the art and commonly used for the treatment of allergy. Antihistamines include, but are not limited to, loratidine, cetirizine, buclizine, ceterizine analogues, fexofenadine, terfenadine, desloratadine, norastemizole, epinastine, ebastine, ebastine, astemizole, levocabastine, azelastine, tranilast, terfenadine, mizolastine, betatastine, CS 560, and HSR 609. Prostaglandin inducers are compounds that induce prostaglandin activity. Prostaglandins function by regulating smooth muscle relaxation. Prostaglandin inducers include, but are not limited to, S-5751.

The asthma/allergy medicaments useful in combination with the IRM compounds also include steroids and immunomodulators. The steroids include, but are not limited to, beclomethasone, fluticasone, tramcinolone, budesonide, corticosteroids and budesonide.

Corticosteroids include, but are not limited to, beclomethasome dipropionate, budesonide, flunisolide, fluticaosone, propionate, and triamcinoone acetonide. Although dexamethasone is a corticosteroid having anti-inflammatory action, it is not regularly used for the treatment of asthma/allergy in an inhaled form because it is highly absorbed, it has long-term suppressive side effects at an effective dose. Dexamethasone, however, can be used according to the invention for the treating of asthma/allergy because when administered in combination with IRM compounds it can be administered at a low dose to reduce the side effects. Additionally, the IRM compounds can be administered to reduce the side effects of dexamethasone at higher concentrations. Some of the side effects associated with corticosteroid include cough, dysphonia, oral thrush (candidiasis), and in higher doses, systemic effects, such as adrenal suppression, osteoporosis, growth suppression, skin thinning and easy bruising. (Barnes &Peterson, Am. Rev. Respir. Dis.; 148: S 1-S26, 1993; and Kamadaet al., Am. J. Respir. Crit Care Med.; 153: 1739-48, 1996).

Systemic corticosteroids include, but are not limited to, methylprednisolone, prednisolone and prednisone. Cortosteroids are associated with reversible abnormalities in glucose metabolism, increased appetite, fluid retention, weight gain, mood alteration, hypertension, peptic ulcer, and rarely asceptic necrosis of femur. These compounds are useful for short-term (3-10 days) prevention of the inflammatory reaction in inadequately controlled persistent asthma. They also function in a long-term prevention of symptoms in severe persistent asthma to suppress and control and actually reverse inflammation. Some side effects associated with longer term use include adrenal axis suppression, growth suppression, dermal thinning, hypertension, diabetes, Cushing's syndrome, cataracts, muscle weakness, and in rare instances, impaired immune function. It is recommended that these types of compounds be used at their lowest effective dose (guidelines for the diagnosis and management of asthma; expert panel report to; NIH Publication No. 97-4051; July 1997).

The immunomodulators include, but are not limited to, the group consisting of anti-inflammatory agents, leukotriene antagonists, IL-4 muteins, soluble IL-4 receptors, immunosuppressants (such as tolerizing peptide vaccine), anti-IL-4antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets. Zileuton tablets function as 5-lipoxygenase inhibitors. These drugs have been associated with the elevation of liver enzymes and some cases of reversible hepatitis and hyperbilirubinemia. Leukotrienes are biochemical mediators that are released from mast cells, eosinophils, and basophils that cause contraction of airway smooth muscle and increase vascular permeability, mucous secretions and activate inflammatory cells in the airways of patients with asthma.

Other immunomodulators include neuropeptides that have been shown to have immunomodulating properties. Functional studies have shown that substance P, for instance, can influence lymphocyte function by specific receptor mediated mechanisms. Substance P also has been shown to modulate distinct immediate hypersensitivity responses by stimulating the generation of arachidonic acid-derived mediators from mucosal mast cells.

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. Anti-IgE is being developed by Genentech. One of skill in the art could prepare functionally active antibody fragments of binding peptides that have the same function. Other types of IgE downregulators are polypeptides capable of blocking the binding of the IgE antibody to the Fc receptors on the cell surfaces and displacing IgE from binding sites upon which IgE is already bound.

One problem associated with downregulators of IgE is that many molecules do not have a binding strength to the receptor corresponding to the very strong interaction between the native IgE molecule and its receptor. The molecules having this strength tend to bind irreversibly to the receptor. However, such substances are relatively toxic since they can bind covalently and block other structurally similar molecules in the body. Of interest in this context is that the a chain of the IgE receptor belongs to a larger gene family where, for example, several of the different IgG Fe receptors are contained. These receptors are absolutely essential for the defense of the body against, for example, bacterial infections. Molecules activated for covalent binding are, furthermore, often relatively unstable and therefore they probably have to be administered several times a day and then in relatively high concentrations in order to make it possible to block completely the continuously renewing pool of IgE receptors on mast cells and basophilic leukocytes.

These types of asthma/allergy medicaments are sometimes classified as long-term control medications or quick-relief medications. Long-term control medications include compounds such as corticosteroids (also referred to as glucocorticoids), methylprednisolone, prednisolone, prednisone, cromolyn sodium, nedocromil, long-acting β₂-agonists, methylxanthines, and leukotriene modifiers. Quick relief medications are useful for providing quick relief of symptoms arising from allergic or asthmatic responses. Quick relief medications include short-acting β₂-agonists, anticholinergics, and systemic corticosteroids.

Chromolyn sodium and medocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from eosinophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratrapoium bromide. These compounds reverse only cholinerigically-mediated bronchospasm and do not modify any reaction to antigen. Side effects include drying of the mouth and respiratory secretions, increased wheezing in some individuals, blurred vision if sprayed in the eyes.

In addition to standard asthma/allergy medicaments other methods for treating asthma/allergy have been used either alone or in combination with established medicaments. One preferred, but frequently impossible, method of relieving allergies is allergen or initiator avoidance. Another method currently used for treating allergic disease involves the injection of increasing doses of allergen to induce tolerance to the allergen and to prevent further allergic reactions.

Allergen injection therapy (allergen immunotherapy) is known to reduce the severity of allergic rhinitis. This treatment has been theorized to involve the production of a different form of antibody, a protective antibody that is termed a “blocking antibody.” Other attempts to treat allergy involve modifying the allergen chemically so that its ability to cause an immune response in the patient is unchanged, while its ability to cause an allergic reaction is substantially altered.

These methods, however, can take several years to be effective and are associated with the risk of side effects such as anaphylactic shock. The use of an IRM compound and asthma/allergy medicament in combination with an allergen avoids many of the side effects etc. Other asthma/allergy medicaments that can be used in the methods and compositions of the invention are listed in U.S. Patent Publication No. U.S. 2003/0087848.

IRM compounds can be combined with other therapeutic agents such as, for example, adjuvants to enhance an immune response. The IRM compound and other therapeutic agent may be administered simultaneously or sequentially. When the other therapeutic agents are administered simultaneously they can be administered in the same or separate formulations, but are administered at the same time. The other therapeutic agents are administered sequentially with one another and with IRM compounds when the administration of the other therapeutic agents and the IRM compound is temporally separated. The separation in time between the administrations of these compounds may be a matter of minutes or it may be longer. Other therapeutic agents include but are not limited to adjuvants, cytokines, antibodies, antigens, etc.

The IRM compounds may be useful as adjuvants for inducing a systemic immune response, a localized immune response, or both. Compositions of the invention also may be administered with one or more non-IRM adjuvants. A non-IRM adjuvant is any molecule or compound except for the IRM compounds described herein that can stimulate a humoral and/or cellular immune response. Non-IRM adjuvants include, for instance, adjuvants that provide controlled release of one or more components of the composition, create a depot effect, immune stimulating adjuvants, or any combination of two or more of the foregoing.

An adjuvant that provides controlled release, as used herein, is an adjuvant that causes the antigen to be slowly released in the body, thus prolonging the exposure of immune cells to the antigen. This class of adjuvants includes but is not limited to alum (e.g., aluminum hydroxide, aluminum phosphate); emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.); polyarginine or polylysine.

An adjuvant that creates a depot effect, as used herein, refers to an adjuvant that is capable of localizing a component of an immunomodulatory combination (e.g., an IRM) so that the component remains where it can most effectively provide the desired effect. In some cases, an adjuvant that creates a depot effect may be covalently attached to a component of the inimunomodulatory combination. For example, attachment of an immunomodulatory combination component to a non-diffusable particle (e.g., a metal or polymeric particle) substantially sequesters the component at the location to which it is delivered, thereby localizing the component and limiting (if not completely preventing) the extent to which the component can diffuse throughout the body. In this way, the activity of the component (e.g., immunomodulatory activity) remains localized to where the component is administered. Thus, (a) activity of the component may be improved by limiting or preventing diffusion of the component, and (b) certain side effects associated with the component (e.g., side effects associated with systemic release of the component) may be reduced or substantially avoided. Examples of such adjuvants include, for example, particles such as those described above and certain lipid-containing moieties. Other adjuvants that create a depot effect are described, for example, in U.S. Provisional Patent Application, Attorney Docket No. 58767US002.

An immune stimulating adjuvant is an adjuvant that causes activation of a cell of the immune system. It may, for instance, cause an immune cell to produce and secrete cytokines. This class of adjuvants includes but is not limited tosaponins purified from the bark of the Q. saponaria tree, such as QS21 (a glycolipid that elutes in the 21st peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi ImmunoChem Research) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Adjuvants that permit controlled release and stimulate the immune system are those compounds which have both of the above-identified functions. This class of adjuvants includes but is not limited to ISCOMS (Immunostimulating complexes that contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals, Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The IRM compounds are also useful as mucosal adjuvants. Other mucosal adjuvants (including nucleic and non-nucleic acid mucosal adjuvants) may also be administered with the IRM compounds. A non-nucleic acid mucosal adjuvant as used herein is an adjuvant other than an immunostimulatory nucleic acid that is capable of inducing a mucosal immune response in a subject when administered to a mucosal surface in conjunction with an antigen. Mucosal adjuvants include but are not limited to bacterial toxins such as, for example, cholera toxin (CT), CT derivatives including but not limited to CTB subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN₁O₇ (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Serto Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler B D, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi, outer membrane protine of Neisseria meningitidis) (Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worcester, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micelle-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Immune responses can also be induced or augmented by the co-administration or co-linear expression of cytokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al., 1997) with the IRM compounds. The cytokines can be administered directly with IRM compounds or may be administered in the form of a nucleic acid vector that encodes the cytokine, such that the cytokine can be expressed in vivo. In one embodiment, the cytokine is administered in the form of a plasmid expression vector. The term cytokine is used as a generic name for a diverse group of soluble proteins and peptides that act as humoral regulators at nanomolar to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), IFN-α, IFN-γ, TNF, transforming growth factor beta (TGF-β), FLT-3 ligand, and CD40 ligand.

The compositions and methods of the invention can be used to modulate an immune response. The ability to modulate an immune response allows for the prevention and/or treatment of particular disorders that can be affected via immune system modulation.

Therapeutic treatment of a disorder aims to reduce, ameliorate or altogether eliminate the disorder, and/or its associated symptoms, or prevent it from becoming worse. Prophylactic treatment of a disorder has aims to reduce the risk of developing the disorder. As used herein, the term “prevent” refers to the prophylactic treatment of patients who are at risk of developing a disorder (resulting in a decrease in the probability that the subject will develop the disorder), and to the inhibition of further development of an already established disorder.

Combined with the teachings provided herein, by choosing among the various active compounds and weighing factors such as potency, relative bioavailability, patient body weight, severity of adverse side effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular IRM compound or other therapeutic agent being administered (e.g., in the case of an immunostimulatory nucleic acid, the type of nucleic acid, i.e., a CpG nucleic acid, the number of unmethylated CpG motifs or their location in the nucleic acid, the degree of modification of the backbone to the oligonucleotide, etc.), the size of the subject, or the severity of the disease or condition. One of ordinary skill in the art can empirically determine the effective amount of a particular IRM compound and/or other therapeutic agent without necessitating undue experimentation.

The term “effective amount” of an IRM compound refers to the amount necessary or sufficient to realize a desired biologic effect. In general, an effective amount of an IRM compound is that amount necessary to cause activation of the immune system, resulting potentially in the development of an antigen specific immune response. In some embodiments, the IRM compounds are administered in an effective amount to stimulate or induce a TH 1 immune response or a general immune response. An effective amount to stimulate a T_{H}1 immune response may be defined as that amount which stimulates the production of one or more T_{H}1-type cytokines such as, for example, IL-2, IL-12, TNF-α, and IFN-γ, and/or production of one or more T_{H}1-type antibodies.

According to some aspects of the invention, an effective amount is that amount of an IRM compound and that amount of another therapeutic agent, such as an antibody, an antigen, an immunostimulatory nucleic acid or a disorder-specific medicament which when combined or co-administered, results in a synergistic response. A synergistic amount is that amount which produces a response that is greater than the sum of the individual effects of the IRM compound and the other therapeutic(s) alone.

An effective amount of an IRM compound is an amount sufficient to induce or increase at least one biological activity associated with increasing an immune response such as, for example, the biological activities described above. The precise amount of IRM compound for increasing a subject's immune response will vary according to factors known in the art including but not limited to the physical and chemical nature of the IRM compound, the nature of the carrier, the intended dosing regimen, the state of the subject's immune system (e.g., suppressed, compromised, stimulated), the method of administering the IRM compound, the nature and immunomodulating potency of other components of the immunomodulatory combination, and the species to which the formulation is being administered. Accordingly, it is not practical to set forth generally the amount that constitutes an effective amount of IRM compound for all possible applications. Those of ordinary skill in the art, however, can readily determine the appropriate amount with due consideration of such factors.

In some embodiments, the methods of the present invention include administering sufficient IRM compound to provide a dose of, for example, from about 100 ng/kg to about 50 mg/kg to the subject, although in some embodiments, the methods may be performed by administering IRM compound in concentrations outside this range. In some embodiments, the method includes administering sufficient IRM compound to provide a dose of from about 10 μg/kg to about 5 mg/kg to the subject, for example, a dose of from about 100 μg/kg to about 1 mg/kg.

An effective amount a therapeutic agent (e.g., an antibody, an antigen, an immunostimulatory nucleic acid, or a disorder-specific medicament) is an amount sufficient to induce or increase at least one biological activity associated with increasing an immune response such as, for example, the biological activities described above. The precise amount of therapeutic agent for increasing a subject's immune response will vary according to factors known in the art including but not limited to the physical and chemical nature of the therapeutic agent, the nature of the carrier, the intended dosing regimen, the state of the subject's immune system (e.g., suppressed, compromised, stimulated), the nature and immunomodulating potency of other components of the immunomodulatory combination, and the species to which the formulation is being administered. Accordingly, it is not practical to set forth generally the amount that constitutes an effective amount of all suitable therapeutic agents for all possible applications. Those of ordinary skill in the art, however, can readily determine the appropriate amount with due consideration of such factors.

As an example, a synergistic combination of an IRM compound and a cancer medicament provides a biological effect which is greater than the combined biological effect which could have been achieved using each of the components (i.e., the agent and the medicament) separately. The biological effect may be the amelioration and or absolute elimination of symptoms resulting from the cancer. In another embodiment, the biological effect is the complete abrogation of the cancer, as evidenced for example, by the absence of a tumor or a biopsy or blood smear that is free of cancer cells.

As another example, an effective amount of an IRM compound and an asthma/allergy medicament is that amount necessary to prevent the development of IgE, or to cause a reduction in IgE levels, or to cause the shift to a T_{H}1 response, in response to an allergen or initiator. In other embodiments, the physiological result is a shift from T_{H}2 cytokines, such as IL-4 and IL-5, to T_{H}1 cytokines, such as IFN-γ and IL-12.

In order to determine the effective amount of IRM compound can be determined using in vitro stimulation assays. The stimulation index of the IRM compound can be compared to that of previously tested IRM compounds and/or certain immunostimulatory acids. The stimulation index can be used to determine an effective amount of the particular IRM compound for the particular subject, and the dosage can be adjusted upwards or downwards to achieve the desired levels in the subject. Effective amounts of IRM compounds can also be determined from animal models, or from human clinical trials using IRM compounds and for compounds that are known to exhibit similar pharmacological activities, such as immunostimulatory nucleic acids and adjuvants, e.g., LT and other antigens for vaccination purposes.

In some instances, a sub-therapeutic dosage of either the IRM compound or the other therapeutic agent, or a sub-therapeutic dosage of both, is used in the treatment of a subject having, or at risk of developing, a disorder. As an example, it has been discovered according to the invention, that when the two classes of drugs are used together, the medicament can be administered in a sub-therapeutic dose and still produce a desirable therapeutic result. A “sub-therapeutic dose” as used herein refers to a dosage that is less than that dosage which would produce a therapeutic result in the subject if administered in the absence of the other agent. Therapeutic doses of certain medicaments are well known in the field of medicine and these dosages have been extensively described in references such as Remington's Pharmaceutical Sciences, 18th ed., 1990; as well as many other medical references relied upon by the medical profession as guidance. Therapeutic dosages of IRM compounds have also been described in the art and methods for identifying therapeutic dosages in subjects are described in more detail herein.

In other aspects, the method of the invention involves administering a high dose of a disorder-specific medicament to a subject, while reducing side effects associated with such a high dose of the medicament so that the side effects are more tolerable. Ordinarily, when a medicament is administered in a high dose, a variety of side effects can occur, as discussed in more detail above, as well as in the medical literature. As a result of these side effects, the medicament is not administered in such high doses, no matter what therapeutic benefits are derived. Such high doses of medicaments, which ordinarily induce an undesirable level of side effects, can be administered with an IRM compound to make the side effects more tolerable. The type and extent of the side effects ordinarily induced by the medicament will depend on the particular medicament used. Examples of immunomodulatory combinations that include an IRM compound to reduce side effects associated with various primary treatments are described in, for example, U.S. Provisional Patent Application Ser. No. 60/526,240, filed Dec. 2, 2003.

Administration of the IRM compound can occur prior to, concurrently with, or following administration of the antibody. If the IRM compound is administered prior to the antibody, typically there is a 1 to 7 day interval between the administrations. If the IRM compound is administered following the antibody, typically there is a 2-3 day interval between the administrations.

In some embodiments of the invention, the IRM compound may be administered once, although in some embodiments the invention may be practiced by administering the IRM compound more than once. In embodiments of the invention in which the IRM compound is administered on a routine schedule. The other therapeutic agents including antibodies, antigens, immunostimulatory nucleic acids and disorder-specific medicaments also may be administered on a routine schedule, but alternatively, may be administered as symptoms arise.

A “routine schedule” as used herein, refers to a predetermined designated period of time. The routine schedule may encompass periods of time which are identical or which differ in length, as long as the schedule is predetermined. For instance, the routine schedule may involve administration on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration on a daily basis for the first week, followed by a monthly basis for several months, and then every three months after that. Any particular combination would be covered by the routine schedule as long as it is determined ahead of time that the appropriate schedule involves administration on a certain day.

In methods particularly directed at subjects at risk of developing a disorder, timing of the administration of the IRM compound and the disorder-specific medicament may also be particularly important. For instance, in a subject with a genetic predisposition to cancer, the IRM compound and the cancer medicament, preferably in the form of an immunotherapy or a cancer medicament, may be administered to the subject on a regular basis.

In some aspects of the invention, the IRM compound is administered to the subject in anticipation of an asthmatic or allergic event in order to prevent an asthmatic or allergic event. The asthmatic or allergic event may be, but need not be limited to, an asthma attack, seasonal allergic rhinitis (e.g., hay-fever, pollen, ragweed hypersensitivity) or perennial allergic rhinitis (e.g., hypersensitivity to allergens such as those described herein). In some instances, the IRM compound is administered substantially prior to an asthmatic or an allergic event. As used herein, “substantially prior” means at least six months, at least five months, at least four months, at least three months, at least two months, at least one month, at least three weeks, at least two weeks, at least one week, at least 5 days, or at least 2 days prior to the asthmatic or allergic event.

Similarly, the asthma/allergy medicament may be administered immediately prior to the asthmatic or allergic event (e.g., within 48 hours, within 24 hours, within 12 hours, within 6 hours, within 4 hours, within 3 hours, within 2 hours, within 1 hour, within 30 minutes or within 10 minutes of an asthmatic or allergic event), substantially simultaneously with the asthmatic or allergic event (e.g., during the time the subject is in contact with the allergen or is experiencing the asthma or allergy symptoms) or following the asthmatic or allergic event.

The immunomodulatory combinations of the invention may be delivered to a particular tissue or cell type or to the immune system or both. In its broadest sense, a “vector” is any vehicle capable of facilitating the transfer of the combinations to the target cells. The vector generally transports the IRM compound, antibody, antigen, immunostimulatory nucleic acid and/or disorder-specific medicament to the target cells with reduced degradation relative to the extent of degradation that would result in the absence of the vector.

In general, the vectors useful in the invention are divided into two classes: biological vectors and chemical/physical vectors. Biological vectors and chemical/physical vectors are useful in the delivery and/or uptake of therapeutic agents of the invention.

Most biological vectors are used for delivery of nucleic acids and this would be most appropriate in the delivery of IRM compounds and targeting agents that are immunostimulatory nucleic acids.

In addition to the biological vectors discussed herein, chemical/physical vectors may be used to deliver IRM compounds and targeting agents, antibodies, antigens, and disorder specific medicaments. As used herein, a “chemical/physical vector” refers to a natural or synthetic molecule, other than those derived from bacteriological or viral sources, capable of delivering the nucleic acid and/or a cancer medicament.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels that are useful as a delivery vector in vivo or in vitro. It has been shown that large unilamellar vessels (LUV), which range in size from about 0.2 μm to about 4.0 μm, can encapsulate large macromolecules. RNA, DNA and intact virions can be encapsulated within the aqueous interior and be delivered to cells in a biologically active form (Fraley, et al., Trends Biochem. Sci., 6: 77, 1981).

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE® (a non-liposomal lipid with a special DNA condensing enhancer) and SUPERFECT® (a novel acting dendrimeric technology).

Liposomes are commercially available from Gibco BRL, for example, as LIPOFECTIN® and LIPOFECTACE®, which are formed of cationic lipids such as N-[1-(2,3 dioleyloxy)-propyl]-N,N,N-trimethylammonium chloride (DOTMA) and dimethyldioctadecylammonium bromide (DDAB). Methods for making liposomes are well known in the art and have been described in many publications.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System.” PCT/US/03307 describes a biocompatible, preferably biodegradable polymeric matrix for containing an exogenous gene under the control of an appropriate promoter. The polymeric matrix can be used to achieve sustained release of the IRM compound and/or the cancer medicament in the subject.

The polymeric matrix preferably is in the form of a microparticle such as a microsphere (wherein the IRM compound and/or the other therapeutic agent is dispersed throughout a solid polymeric matrix) or a microcapsule (wherein the IRM compound and/or the other therapeutic agent is stored in the core of a polymeric shell). Other forms of the polymeric matrix for containing the IRM compound and/or the other therapeutic agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the other therapeutic agent are encompassed in a surfactant vehicle. The polymeric matrix composition can be selected to have both favorable degradation rates and also to be formed of a material that is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the IRM compounds are administered to the subject via an implant while the other therapeutic agent is administered acutely. Biocompatible microspheres that are suitable for delivery, such as oral or mucosal delivery are disclosed in Chickering et al., Biotech. And Bioeng., (1996) 52: 96-101 and Mathiowitz et al., Nature, (1997) 386: 410-414 and PCT Patent Application WO97/03702.

Both non-biodegradable and biodegradable polymeric matrices can be used to deliver the IRM compound and/or other therapeutic agent to the subject. Biodegradable matrices are preferred. Such polymers may be natural or syntheticpolymers. The polymer is selected based on the period of time over which release is desired, generally in the order of a few hours to a year or longer. Typically, release over a period ranging from between a few hours and three to twelve months is most desirable, particularly for the IRM compounds. The polymer optionally is in the form of a hydrogel that can absorb up to about 90% of its weight in water and further, optionally is cross-linked with multi-valent ions or other polymers.

Bioadhesive polymers of particular interest include bioerodible hydrogels such as those described, for example, in H. S. Sawhney, C. P. Pathak and J. A. Hubell in Macromolecules, (1993) 26: 581-587, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

If the therapeutic agent is a nucleic acid, the use of compaction agents may also be desirable. Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of a nucleic acid include calcium phosphate and other chemical mediators of intracellular transport, microinjection compositions, electroporation and homologous recombination compositions (e.g., for integrating a nucleic acid into a preselected location within the target cell chromosome).

The compounds may be administered alone (e.g. in saline or buffer) or using any delivery vectors known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically acceptable carrier means one or more compatible solid or liquid filler, diluents, or encapsulating substances that are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction that would substantially impair the desired pharmaceutical efficiency.

The IRM compounds useful in the invention may be delivered in mixtures with additional adjuvant(s), other therapeutics, or antigen(s). A mixture may consist of several adjuvants in addition to the IRM compound or several antigens or other therapeutics.

The IRM compounds and other compounds can be administered by any ordinary route for administering medications. A variety of administration routes are available. The particular mode selected will depend, of course, upon the particular adjuvants or antigen selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed herein. For use in therapy, an effective amount of the IRM compound can be administered to a subject by any mode that delivers the agent to the desired surface, e.g., mucosal, systemic.

Administering the pharmaceutical composition of the present invention may be accomplished by any means known to the skilled artisan. Preferred routes of administration include but are not limited to oral, parenteral, intramuscular, intranasal, intratracheal, inhalation, ocular, vaginal, and rectal. For the treatment or prevention of asthma or allergy, such compounds are preferably inhaled, ingested or administered by systemic routes. Systemic routes include oral and parenteral. Inhaled medications are preferred in some embodiments because of the direct delivery to the lung, the site of inflammation, primarily in asthmatic patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

Components of an immunomodulatory combination may be provided in any formulation suitable for administration to a subject. Suitable types of formulations are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,186; European Patent No. EP 0 394 026; and International Patent Publication No. WO 03/045391. Each component may be provided in any suitable form including but not limited to a solution, a suspension, an emulsion, or any form of mixture. Suitable formulations may include any pharmaceutically acceptable excipient, carrier, or vehicle.

In some embodiments, the methods of the present invention include administering each component of the immunomodulatory combination to a subject in a formulation of, for example, from about 0.0001% to about 10% (unless otherwise indicated, all percentages provided herein are weight/weight with respect to the total formulation) to the subject, although in some embodiments, each component may be administered using a formulation that provides one or more components in a concentration outside of this range. In certain embodiments, the method includes administering to a subject a formulation that includes at least about 0.001% IRM compound. In certain specific embodiments, the method includes administering to a subject a formulation that includes at least about 0.01% IRM compound, for example, at least about 0.1% IRM compound. In some embodiments, the IRM compound may be provided at a concentration of up to about 5%. In certain embodiments, the IRM compound may be provided at a concentration of up to about 1%, for example, up to about 0.5%.

For oral administration, the compounds (i.e., IRM compounds, antigens, antibodies, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a controlled release preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems.

The IRM compounds and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkoniumchloride (0.003-0.03% w/v); chlorobutanol (0.3-0.9% w/v); parabens (0.01-0.25% w/v) and thimerosal (0.004-0.02% w/v).

The compositions may conveniently be presented in unit dosage form and may be prepared by any of the methods well known in the art of pharmacy. All methods include the step of bringing the compounds into association with a carrier that includes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories. For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units. Multiple administration of doses at specific intervals of weeks or months apart is usual for boosting the antigen-specific responses.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775; 4,675,189; and 5,736,152, and (b) diffusional systems in which an active component permeates at a controlled rate from a polymer such as described in U.S. Pat. Nos. 3,854,480; 5,133,974; and 5,407,686. In addition, pump-based hardware delivery systems can be used, some of which are adapted for implantation.

In other aspects of the invention, a composition is provided. The composition includes an IRM compound and another therapeutic agent formulated in a pharmaceutically acceptable carrier and present in the composition in an effective amount.

In other aspects, the invention relates to kits. One kit of the invention includes a sustained release vehicle containing an IRM compound and a container housing another therapeutic agent and instructions for timing of administration of the compounds. A sustained release vehicle is used herein in accordance with its prior art meaning of any device which slowly releases the compound contained therein.

The container may be a single container housing all of a medicament together or it may be multiple containers or chambers housing individual dosages of the medicament, such as a blister pack. The kit also has instructions for timing of administration of the medicament. The instructions would direct the subject to take the medicament at the appropriate time. For instance, the appropriate time for delivery of the medicament may be as the symptoms occur. Alternatively, the appropriate time for administration of the medicament may be on a routine schedule such as monthly or yearly.

Another kit of the invention includes at least one container housing an IRM compound and at least one container housing another therapeutic agent and instructions for administering the compositions in effective amounts for inducing a synergistic immune response in the subject. The instructions in the kit may direct the subject to take compounds in amounts that will produce a synergistic immune response. The drugs may be administered simultaneously or separately as long as they are administered close enough in time to produce a synergistic response.

In some embodiments, the IRM compound of an immunomodulatory combination may be a compound identified as an agonist of one or more TLRs. In some embodiments, the IRM compound can act as an agonist of one or more of TLR6, TLR7, or TLR8. The IRM may also in some cases be an agonist of TLR 9. In some embodiments, the IRM compound may be an agonist of TLR7 such as, for example, a TLR7-selective agonist. In other embodiments, the IRM compound may be a TLR8 agonist such as, for example, a TLR8-selective agonist. In still other embodiments, the IRM compound may be a TLR7/8 agonist.

As used herein, the term “TLR8-selective agonist” refers to any compound that acts as an agonist of TLR8, but does not act as an agonist of TLR7. A “TLR7-selective agonist” refers to a compound that acts as an agonist of TLR7, but does not act as an agonist of TLR8. A “TLR7/8 agonist” refers to a compound that acts as an agonist of both TLR7 and TLR8.

A TLR8-selective agonist or a TLR7-selective agonist may act as an agonist for the indicated TLR and one or more of TLR1, TLR2, TLR3, TLR4, TLR5, TLR6, TLR9, or TLR10. Accordingly, while “TLR8-selective agonist” may refer to a compound that acts as an agonist for TLR8 and for no other TLR, it may alternatively refer to a compound that acts as an agonist of TLR8 and, for example, TLR6. Similarly, “TLR7-selective agonist” may refer to a compound that acts as an agonist for TLR7 and for no other TLR, but it may alternatively refer to a compound that acts as an agonist of TLR7 and, for example, TLR6.

The TLR agonism for a particular compound may be assessed in any suitable manner. For example, assays for detecting TLR agonism of test compounds are described, for example, in U.S. Patent Publication No. U.S. 2004/0132079, and recombinant cell lines suitable for use in such assays are described, for example, in International Patent Publication No. WO04/053057.

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

Conditions for which the methods described herein may be used as treatments include, but are not limited to:
(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picomavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirussuch as HIV);
(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;
(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and
(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers; and
(e) T_{H}2-mediated, atopic, and autoimmune diseases, such as atopic dermatitis or eczema, eosinophilia, asthma, allergy, allergic rhinitis, systemic lupus erythematosus, essential thrombocythaemia, multiple sclerosis, Ommen's syndrome, discoid lupus, alopecia greata, inhibition of keloid formation and other types of scarring, and enhancing would healing, including chronic wounds.

In certain embodiments, an immune response may be desired against a particular antigen such as, for example, an antigen associated with one of the conditions listed above. In such embodiments, the antigen (or at least an immunogenic epitope of the antigen) may be administered to the subject. The antigen may be co-administered with the IRM compound, another therapeutic agent or both. Alternatively, the antigen may be administered separately from the IRM compound and any other therapeutic agent. When the antigen is administered separately, it may be administered before, after, or between the other components of the immunomodulatory combination.

An amount of antigen effective for use in certain embodiments of the invention is an amount sufficient to induce or increase at least one biological activity associated with increasing an immune response such as, for example, the biological activities described above. The precise amount of antigen for increasing a subject's immune response will vary according to factors known in the art including but not limited to the physical and chemical nature of the antigen, the nature of the carrier, the intended dosing regimen, the state of the subject's immune system (e.g., suppressed, compromised, stimulated), the immunomodulatory potency of the IRM compound and other components of the immunomodulatory combination, and the species to which the formulation is being administered. Accordingly, it is not practical to set forth generally the amount that constitutes an effective amount of antigen for all possible applications. Those of ordinary skill in the art, however, can readily determine the appropriate amount with due consideration of such factors.

In some embodiments, the methods of the present invention include administering sufficient antigen to provide a dose of, for example, from about 100 ng/kg to about 50 mg/kg to the subject, although in some embodiments the methods may be performed by administering antigen in concentrations outside this range. In some of these embodiments, the method includes administering sufficient antigen to provide a dose of from about 10 μg/kg to about 5 mg/kg to the subject, for example, a dose of from about 100 μg/kg to about 1 mg/kg.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The IRM compounds used in the examples are shown in Table 1.

TABLE 1

| Compound | Chemical Name | Reference |
|---|---|---|
| IRM 1 | 1-(2-methylpropyl)-1H-imidazo[4,5-c] | U.S. Pat. No. 6,194,425 |
| | [1,5]naphthyridin-4-amine | Example 32 |
| IRM 2 | 2-methyl-1-(2-methylpropyl)-1H-imidazo[4,5-c] | U.S. Pat. No. 6,194,425 |
| | [1,5]naphthyridin-4-amine | Example 36 |
| IRM3 | N-{2-[4-amino-2-(ethoxymethyl)-1H-imidazo[4,5- | U.S. Pat. No. 6,677,349 |
| | c]quinolin-1-yl]-1,1- | Example 268 |
| | dimethylethyl}methanesulfonamide | |
| IRM4 | 4-amino-α,α,2-trimethyl-1H-imidazo[4,5- | U.S. Pat. No. 5,266,575 |
| | c]quinolin-1-ethanol | Example C1 |
| IRM5 | N-(2-{2-[4-amino-2-(2-methoxyethyl)-1H- | U.S. Pat. No. 6,656,938 |
| | imidazo[4,5-c]quinolin-1-yl]ethoxy}ethyl)-N- | Example 6 |
| | methylmorpholine-4-carboxamide | |

Table 2 shows the formulation of the vehicle used in the following examples, on a percentage weight-by-weight basis.

TABLE 2

| Materials | Vehicle (w/w %) |
|---|---|
| Isopropyl Myristate, NF | 10.00 |
| Isostearic Acid | 5.00 |
| Poloxamer 188, NF | 2.5 |
| Disodium EDTA, USP | 0.05 |
| Carbomer 974, NF | 1.00 |
| Propylene glycol, USP | 15.00 |
| Propylparaben, NF | 0.1 |
| Methylparaben, NF | 0.2 |
| Purified water, USP | 65.65 |
| 20% w/w NaOH | 0.5 |

Formulations containing IRM compound were prepared by adding the appropriate amount of IRM compound, on a percentage weight-by-weight basis, to the vehicle to obtain the final IRM weight percentage, and decreasing the amount of water added accordingly.

The formulation was prepared as follows:

Oil phase preparation: IRM compound, when present, was dissolved in isostearic acid and isopropyl myristate, with heat if necessary. Carbomer 974 was then dispersed in the oil phase.

Water phase preparation: Disodium EDTA was dissolved in the water. Methylparaben and propylparaben were dissolved in propylene glycol and the solution was subsequently added to the water phase. Poloxamer 188 was then added to the water phase and mixed until dissolved.

Phase combination: The oil phase was added to the water phase at ambient conditions. The emulsion was then homogenized. After homogenization, sodium hydroxide solution (20% w/w) was added and the resulting cream was mixed until smooth and uniform. The pH of the cream was measured and a pH adjustment was made with additional sodium hydroxide solution, if necessary, to meet the in-process target pH of 5.0.

Female SKH-1 mice 9-10 weeks old were obtained from Charles River (Raleigh, N.C.) and housed in a room with controlled temperatures and humidity and alternating 12-hour light and dark cycles. The room is lit with fluorescent lights covered by yellow sleeves from EncapSulite International, Inc. (Rosenberg, Tex.) to eliminate all ambient UV radiation. The mice were fed with a 12% Corn Oil Purified Diet from Purina Test Diet (Richmond, Va.) and water, ad libitum. The animals were maintained in facilities approved by the Association for the Assessment and Accreditation of Laboratory Animal Care International and in accordance with current United States Department of Agriculture, Department of Health and Human Services, and National Institutes of Health regulations and standards.

A small group of mice were not exposed to UV and were used as negative controls. The other mice were chronically UV irradiated. After randomization the mice were lightly anesthetized with halothane and adjustable collars made of hook and loop fastener approximately 10 mm wide and 70 mm long were placed around the mouse's neck to prevent oral ingestion of the IRM. Thirty minutes before UV exposure mice were topically dosed with 30 μL of either (1) vehicle formulation, (2) IRM formulation, or (3) an SPF 30 sunscreen (NEUTROGENA SPF30 Sunblock with Parsol 1789), two times per week for a total of 15-18 weeks on the dorsal surface in an area approximately 6 cm². Approximately four hours after dosing, the treatment (i.e, vehicle formulation, IRM formulation, or sunscreen) was washed off using a dilute soap solution.

Mice were placed in a standard plexiglass rat cage, 9-10 at a time, that was separated into 12 individual compartments with plexiglass dividers and placed on a shelf 14 inches below the light source without wire cage tops. The mice were exposed to UV radiation 5 times per week (Monday thru Friday) for an average of 15-18 weeks. The UV radiation was provided by a bank of six FS40 lamps (National Biological Corporation, Twinsburg, Ohio), filtered by a Kodacel filter (140 μm thick K6808 cellulose triacetate film; Eastman Kodak, Rochester, N.Y.) that transmits radiation having a wavelength from about 293 nm to about 400 nm. The Kodacel filter was aged 4-6 hours before use. The lights emit UVB (21%) and UVA (79%) light with a peak wavelength of 313 nm, as measured using a radiometer Model PMA 2200 from Solar Light Company (Philadelphia, Pa.) with a PMA 2101 detector for UVB and PMA2110 detector for UVA.

The dose of UVB irradiation was measured by MED (minimal erythemal dose). By using MED, a relatively constant incident dose of UV was maintained over time. The initial dose of UVB to the mice was 12.6 mJ/cm² (0.6 MED) and the average total dose of UVB was 3000-4500 mJ/cm². An increase of 10-20% of the MED dose per week was needed due to acclimation of the mice.

All mice were distinguished from one another by a tattoo placed on the tail. The development and appearance of tumors were noted weekly and recorded starting at about 14-18 weeks.

### Example 1

UV dosing and IRM dosing were initiated simultaneously. UV dosing was performed five days per week for 15 weeks. Dosing was performed twice per week at the doses indicated in Table 3.

After 15 weeks, the mice were subjected to gross inspection to determine the presence of a disease state, i.e., squamous cell carcinoma (SCC), actinic keratosis (AK), or pre-AK lesions. Results are shown in Table 3 and are expressed as the percentage of mice exhibiting a disease state:

$$\% Disease = \frac{( SCC + AK + pre \text{-} AK lesion )}{Total mice}$$

TABLE 3

| Treatment | No. of mice | % Disease |
|---|---|---|
| UV Exposed - vehicle | 10 | 70 |
| UV Exposed - 0.1% IRM 1 | 10 | 0 |
| UV Exposed - 0.1% IRM 2 | 9 | 11 |
| No UV exposure | 5 | 0 |

### Example 2

UV dosing was performed for six weeks before IRM dosing was initiated. UV dosing was performed five days per week for 15 weeks. Dosing was performed twice per week at the doses indicated in Table 4.

After 15 weeks, the mice were subjected to gross inspection to determine the presence of a disease state, i.e., squamous cell carcinoma (SCC), actinic keratosis (AK), or pre-AK lesions. Results are shown in Table 4.

TABLE 4

| Treatment | No. of mice | % Disease |
|---|---|---|
| UV Exposed - 0.1% IRM 2 | 8 | 63 |
| UV Exposed - 1.0% IRM 2 | 6 | 50 |
| UV Exposed - SPF 30 sunscreen | 8 | 63 |
| No UV exposure | 5 | 0 |

### Example 3

UV dosing and IRM dosing were initiated simultaneously. UV dosing was performed five days per week for 18 weeks. Dosing was performed twice per week at the doses indicated in Table 5.

After 18 weeks, the mice were subjected to gross inspection to determine the presence of a disease state, i.e., squamous cell carcinoma (SCC), actinic keratosis (AK), or pre-AK lesions. Results are shown in Table 6.

The disease state of each mouse was scored by measuring the size and/or number of lesions present on the mouse. SCC lesions and AK lesions of at least 1 mm in diameter were given a score of 1. Pre-AK lesions of less than 1 mm were scored as indicated in Table 5.

TABLE 5
Scoring of UV-induced lesions less than 1 mm in diameter

| Lesion Frequency | Score |
|---|---|
| Rare (0-1) | 0 |
| Few (2-10) | 0.5 |
| Moderate (11-20) | 1.0 |
| Many (>21) | 1.5 |

The SCC lesion score, AK lesion score, and the pre-AK lesion score (according to Table 5) were added to provide a total disease score for each mouse. Table 6 includes the average disease score for all of the mice in each treatment group.

TABLE 6

| | No. of | % | |
|---|---|---|---|
| Treatment | Mice | Disease | Avg. Disease Score |
| UV Exposed - 0.1% IRM 2 | 8 | 25 | 0.6 ± 0.42 |
| UV Exposed - 0.01% IRM 2 | 7 | 71 | 4.86 ± 1.81 |
| UV Exposed - 0.1% IRM 1 | 6 | 50 | 0.66 ± 0.33 |
| UV Exposed - 0.01% IRM 1 | 7 | 71 | 2.86 ± 0.82 |
| UV Exposed - SPF 30 sunscreen | 5 | 40 | 0.6 ± 0.4 |
| UV Exposed - vehicle | 8 | 75 | 1.75 ± 0.53 |
| No UV exposure | 5 | 0 | 0 |

### Example 4

IRM3 was prepared as a 0.375% solution formulation capable of being nasally administered via a spray pump. The formulation vehicle was prepared as follows:

TABLE 7

| Excipient | w/w % |
|---|---|
| Carboxymethyl cellulose, USP (Spectrum Chemicals and | 0.1 |
| Laboratory Products, Inc., Gardena, CA,) | |
| Benzalkonium chloride, Ph. Eur. (Fluka, Buchs Switzerland) | 0.02 |
| Disodium EDTA, USP (Spectrum Chemicals) | 0.1 |
| L-Lactic acid, Purac (Lincolnshire, IL) | 1.53 |
| PEG 400, NF (Spectrum Chemicals) | 15 |
| 1 N NaOH, NF (Spectrum Chemicals) | qs |
| Water | qs |
| Total | 100.00 |
| pH | 4.0 |

Carboxymethyl cellulose (CMC) was hydrated in water (about 50% of total) for 20 minutes with stirring. The EDTA was added and dissolved. The CMC/EDTA solution was mixed with the benzalkonium chloride to form a CMC/EDTA/BAC solution. Separately, the lactic acid and PEG 400 were mixed with water. For the IRM formulation, IRM3 was dissolved into the lactic acid/PEG 400 solution. The CMC/EDTA/BAC solution was mixed with lactic acid/PEG 400 solution to prepare the Vehicle formulation. The CMC/EDTA/BAC solution was mixed with lactic acid/PEG 400/IRM solution to prepare the IRM formulation. 1 N NaOH was added, as necessary, to adjust each formulation to a pH of 4.0. Finally, water was added to each formulation to adjust to the final formulation weight.

### Example 5

Fisher 344 rats (Charles River Laboratories, Raleigh, N.C.) were divided into six treatment groups. Rats in each group were infected intranasally with humanized, non-lethal influenza virus. 24 hours after infection, viral titers were measured in nasal lavage fluid and whole lung homogenates. The influenza virus and methods for measuring viral titers are described in Burleson, Gary L., “Influenza Virus Host Resistance Model for Assessment of Immunotoxicity, Immunostimulation, and Antiviral Compounds,” Methods in Immunology 2: 181-202, Wiley-Liss Inc., 1995.

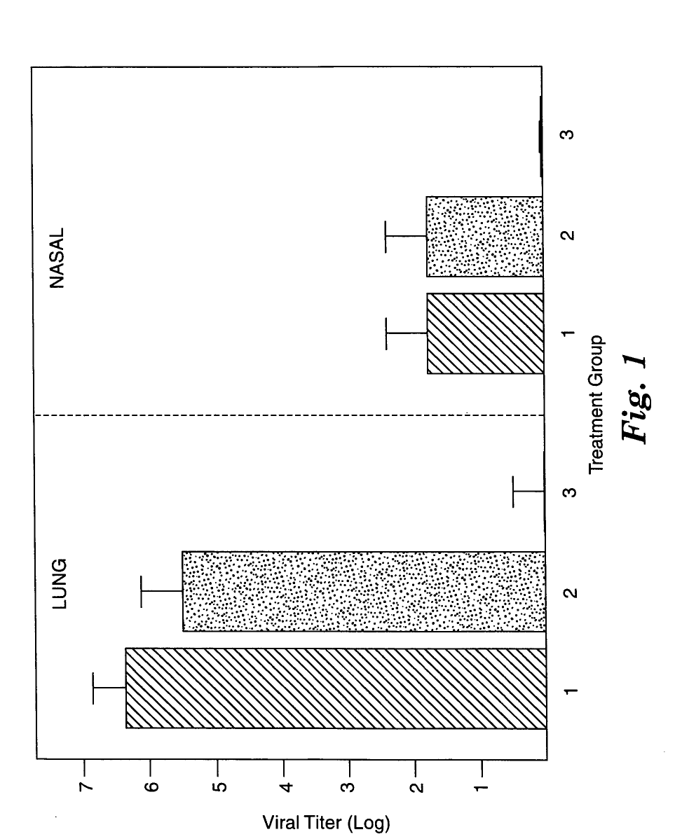

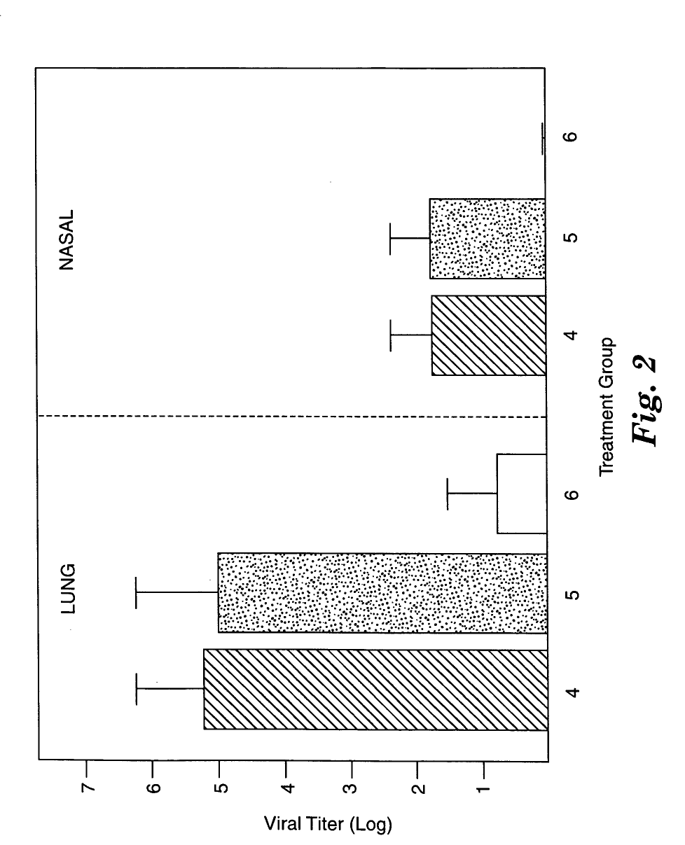

Each of the six treatment groups received a different pre-infection treatment. Rats in each group received the treatment indicated in Table 8. The results are summarized in FIG. 1 and FIG. 2.

TABLE 8

| Group | Treatment |
|---|---|
| 1 | Vehicle formulation (Table 1), 50 μL (25 μL per nare), 1x* |
| 2 | Interferon-α (rat recombinant IFN-α, Cat. No. PRP13, |
| | Serotec Inc., Raleigh, NC), 10,000 IU, 1x |
| 3 | IRM formulation (Table 1), 50 μL (25 μL per nare), 1x |
| 4 | Vehicle formulation (Table 1), 50 μL (25 μL per nare), 2x** |
| 5 | Interferon-α, 10,000 IU, 2x (Day −1: Product No. RR2030U, |
| | Pierce Biotechnology, Inc., Rockford, IL; Day 0: Serotec Inc. |
| | Cat. No. PRP13) |
| 6 | IRM formulation (Table 1), 50 μL (25 μL per nare), 2x |

*1x: one dose of treatment provided four hours before viral infection.
**2x: one dose of treatment 24 hours (Day −1) before viral infection, second treatment four hours before viral infection (Day 0).

### Example 6

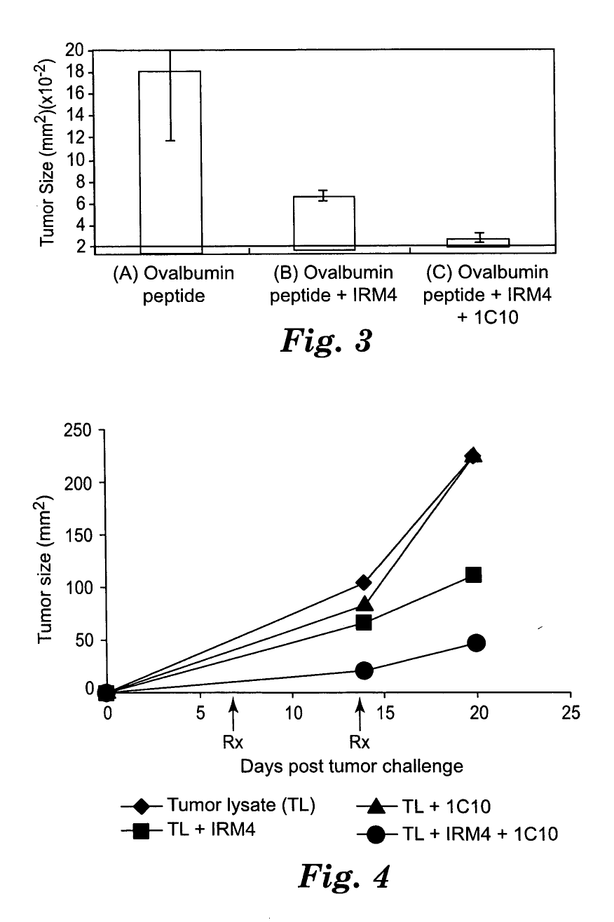

Mice were challenged intradermally on day 0 with 1×10⁵ melanoma B16ova tumor cells in PBS (Kedl et al. PNAS 98(19): 10811-10816). On day 7, the mice were immunized with either (A) 100 μg ovalbumin peptide, (B) 100 μg ovalbumin peptide+200 μg IRM4, or (C) 100 μg ovalbumin peptide+200 μg IRM4+100 μg 1C10 anti-CD40 antibody. On day 21, the mice were sacrificed and the tumors were measured in two dimensions by caliper. Data are shown in FIG. 3. Immunization with antigen, IRM4 and CD40 agonist resulted in slower tumor growth than immunization with IRM4 alone.

Mice also were challenged as described above, and immunized as described above except that IRM5 was substituted for IRM4. The results observed using IRM5 in place of IRM4 were similar to the results observed using IRM4.

### Example 7

Mice were challenged with tumor on day 0 as in example 6.5 mice each were immunized on days 7 with 1×10⁶ cell equivalents (CE) (A) tumor lysate, (B) 1×10⁶ CE tumor lysate+200 μg IRM4, (C) 1×10⁶ CE tumor lysate+100 μg FGK4.5 anti-CD40 antibody, or (D)1×10⁶ CE tumor lysate+200 μg IRM4+100 μg FGK4.5 anti-CD40 antibody. Tumor sizes were measured on the mice by caliper on days 14 and 20. The data are shown in FIG. 4. Immunization with the combination of IRM4 and anti-CD40 agonists resulted in slower tumor growth than immunization with IRM4 alone or CD40 agonist alone.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A immunomodulatory combination comprising:
an IRM component that comprises an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine, provided in an amount effective, in combination with a therapeutic agent, to induce an immune response in a subject; and
a therapeutic agent in an amount effective, in combination with the IRM component, to induce an immune response in a subject.

2. The immunomodulatory combination of claim 1 wherein the IRM component comprises an imidazonaphthyridine amine, a tetrahydroimidazonaphthyridine amine, an oxazolonaphthyridine amine, or a thiazolonaphthyridine amine.

3. The immunomodulatory combination of claim 1 wherein the therapeutic agent comprises a cancer antigen or a cancer antibody.

4. The immunomodulatory combination of claim 1 wherein the therapeutic agent comprises an antigen of an infectious agent.

5. The immunomodulatory combination of claim 1 wherein the therapeutic component comprises a medicament for treating a T_{H}2-mediated disease.

6. A immunomodulatory combination comprising:
an IRM component comprising in an amount effective, in combination with a therapeutic agent, to induce an immune response in a subject, wherein the IRM component comprises a sulfonamide substituted imidazoquinoline amine, an ether substituted imidazoquinoline amine, a sulfonamide substituted tetrahydroimidazoquinoline amine, an ether substituted tetrahydroimidazoquinoline amine, a sulfonamide substituted imidazopyridine amine, or an ether substituted imidazopyridine amine; and
a therapeutic agent in an amount effective, in combination with the IRM component, to induce an immune response in a subject.

7. The immunomodulatory combination of claim 1 wherein the therapeutic agent comprises a cancer antigen or a cancer antibody.

8. The immunomodulatory combination of claim 1 wherein the therapeutic agent comprises an antigen of an infectious agent.

9. The immunomodulatory combination of claim 1 wherein the therapeutic component comprises a medicament for treating a T_{H}2-mediated disease.
